(19) United States
(12) Patent Application Publication
(10) Pub. No.: US 2022/0376188 A1
(43) Pub. Date: Nov. 24, 2022

(54) ORGANIC LIGHT-EMITTING DEVICE

(71) Applicant: LG CHEM, LTD., Seoul (KR)

(72) Inventors: Minwoo CHOI, Daejeon (KR); Hyeon Soo JEON, Daejeon (KR); Sang Young JEON, Daejeon (KR); Minseung CHUN, Daejeon (KR); Jungha LEE, Daejeon (KR); Jaeeun KIM, Daejeon (KR)

(21) Appl. No.: 17/764,280

(22) PCT Filed: Dec. 11, 2020
(86) PCT No.: PCT/KR2020/018145

§ 371 Date: Mar. 28, 2022

(30) Foreign Application Priority Data
Dec. 11, 2019 (KR) 10-2019-0164734

(51) Int. Cl. H01L 51/00; C07D 209/86; C09K 11/06; C07D 403/14

(52) U.S. Cl. CPC H01L 51/0072; C07D 209/86; C09K 11/06; H01L 51/0052; C07D 403/14; H01L 51/0067; H01L 51/5016

(57) ABSTRACT

Provided is an organic light emitting device including: a first electrode; a second electrode; and an organic material layer having one or more layers provided between the first electrode and the second electrode, in which the organic material layer includes a light emitting layer including a compound of Chemical Formula 1 and a compound of Chemical Formula 2:

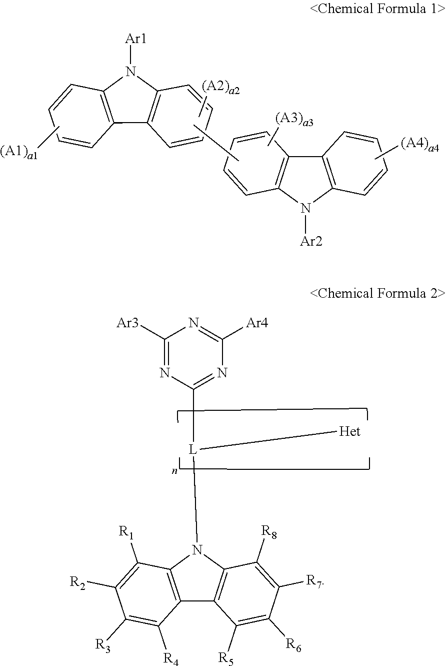

### CROSS-REFERENCE TO RELATED APPLICATIONS

This application is a National Stage Application of International Application No. PCT/KR2020/018145 filed on Dec. 11, 2020, which claims priority to and the benefit of Korean Patent Application No. 10-2019-0164734 filed in the Korean Intellectual Property Office on Dec. 11, 2019, the entire contents of which are incorporated herein by reference.

### TECHNICAL FIELD

The present specification relates to an organic light emitting device including: a first electrode; a second electrode; and an organic material layer having one or more layers provided between the first electrode and the second electrode.

### BACKGROUND

In general, an organic light emitting phenomenon refers to a phenomenon in which electric energy is converted into light energy by using an organic material. An organic light emitting device using the organic light emitting phenomenon usually has a structure including a positive electrode, a negative electrode, and an organic material layer interposed therebetween. Here, the organic material layer has in many cases a multi-layered structure composed of different materials in order to improve the efficiency and stability of the organic light emitting device, and for example, can be composed of a hole injection layer, a hole transport layer, a light emitting layer, an electron transport layer, an electron injection layer, and the like. In such a structure of the organic light emitting device, if a voltage is applied between the two electrodes, holes are injected from the positive electrode into the organic material layer and electrons are injected from the negative electrode into the organic material layer, and when the injected holes and electrons meet each other, an exciton is formed, and light is emitted when the exciton falls down again to a ground state.

There is a continuous need for developing a new material for the aforementioned organic light emitting device.

### BRIEF DESCRIPTION

### Technical Problem

The present specification has been made in an effort to provide an organic light emitting device including: a first electrode; a second electrode; and an organic material layer having one or more layers provided between the first electrode and the second electrode.

### Technical Solution

The present specification provides an organic light emitting device including: a first electrode; a second electrode; and an organic material layer having one or more layers provided between the first electrode and the second electrode,

in which the organic material layer includes a light emitting layer including a compound of the following Chemical Formula 1 and a compound of the following Chemical Formula 2:

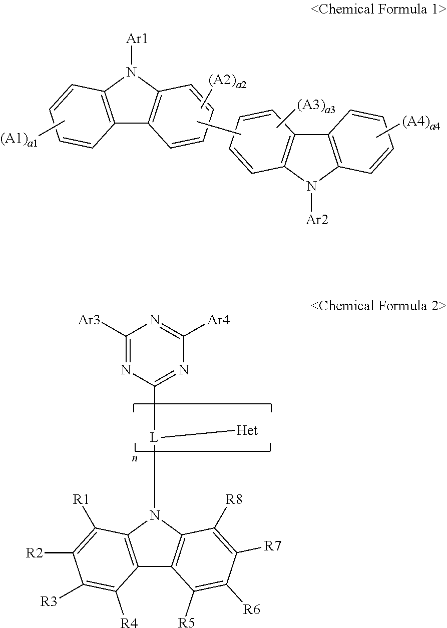

wherein Chemical Formulae 1 and 2:

Ar1 to Ar4 are the same as or different from each other, and are each independently a substituted or unsubstituted aryl group or a substituted or unsubstituted heterocyclic group;

A1 to A4 and R1 to R8 are the same as or different from each other, and are each independently hydrogen, deuterium, a substituted or unsubstituted aryl group, or a substituted or unsubstituted carbazole group, or are bonded to an adjacent group to form a substituted or unsubstituted ring;

Het is a substituted or unsubstituted heterocyclic group having 5 to 20 carbon atoms, which includes N, O, or S;

L is a trivalent aryl group having 5 to 20 carbon atoms;

a1 and a4 are each an integer from 1 to 4, and a2 and a3 are each an integer from 1 to 3;

when a1 to a4 are each an integer of 2 or higher, two or more substituents in the parenthesis are the same as or different from each other;

n is an integer of 0 or 1; and

when n is 0, N of carbazole is directly bonded to triazine.

### Advantageous Effects

When a light emitting layer including the compounds of Chemical Formulae 1 and 2 according to an exemplary embodiment of the present specification is included, the service life of the organic light emitting device is improved.

When a light emitting layer including the compounds of Chemical Formulae 1 and 2 according to an exemplary embodiment of the present specification is included, the organic light emitting device has an advantage of low driving voltage.

### BRIEF DESCRIPTION OF THE DRAWINGS

FIG. 1 illustrates an organic light emitting device 10 according to an exemplary embodiment of the present specification.

FIG. 2 illustrates an organic light emitting device 11 according to another exemplary embodiment of the present specification.

### EXPLANATION OF REFERENCE NUMERALS AND SYMBOLS

10, 11: Organic light emitting device

20: Substrate

30: First electrode

40: Light emitting layer

50: Second electrode

60: Hole injection layer

70: Hole transport layer

80: Electron transport layer

90: Electron injection layer

### DETAILED DESCRIPTION

When one part “includes” one constituent element in the present specification, unless otherwise specifically described, this does not mean that another constituent element is excluded, but means that another constituent element can be further included.

When one member is disposed “on” another member in the present specification, this includes not only a case where the one member is brought into contact with another member, but also a case where still another member is present between the two members.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent can be substituted, and when two or more are substituted, the two or more substituents can be the same as or different from each other.

In the present specification, the term “substituted or unsubstituted” means being substituted with one or two or more substituents selected from the group consisting of deuterium, a halogen group, a nitrile group, a nitro group, a hydroxyl group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, and a substituted or unsubstituted heterocyclic group, or being substituted with a substituent to which two or more substituents are linked among the substituents exemplified above, or having no substituent. For example, “the substituent to which two or more substituents are linked” can be a biphenyl group. That is, the biphenyl group can also be an aryl group, and can be interpreted as a substituent to which two phenyl groups are linked.

In the present specification,

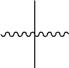

means a moiety bonded to another substituent or a bonding portion.

In the present specification, a halogen group can be fluorine, chlorine, bromine or iodine.

In the present specification, the alkyl group can be straight-chained or branched, and the number of carbon atoms thereof is not particularly limited, but is preferably 1 to 30. Specific examples thereof include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present specification, a cycloalkyl group is not particularly limited, but has preferably 3 to 30 carbon atoms, and specific examples thereof include cyclopropyl, cyclobutyl, cyclopentyl, 3-methylcyclo-pentyl, 2,3-dimethylcyclopentyl, cyclohexyl, 3-methyl-cyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present specification, the alkoxy group can be straight-chained, branched, or cyclic. The number of carbon atoms of the alkoxy group is not particularly limited, but is preferably 1 to 30. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, an amine group can be selected from the group consisting of —NH₂; an alkylamine group; an N-alkylarylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, a triphenylamine group, an N-phenylbiphenylamine group; an N-phenylnaphthylamine group; an N-biphenyl-naphthylamine group; an N-naphthylfluorenylamine group; an N-phenyl-phenanthrenylamine group; an N-biphenyl-phenanthrenylamine group; an N-phenylfluorenylamine group; an N-phenyl terphenylamine group; an N-phenanthrenylfluorenylamine group; an N-biphenyl-fluorenylamine group, and the like, but are not limited thereto.

In the present specification, an N-alkylarylamine group means an amine group in which an alkyl group and an aryl group are substituted with N of the amine group.

In the present specification, an N-arylheteroaryl-amine group means an amine group in which an aryl group and a heteroaryl group are substituted with N of the amine group.

In the present specification, an N-alkyl-heteroarylamine group means an amine group in which an alkyl group and a heteroaryl group are substituted with N of the amine group.

In the present specification, the alkyl group in the arylalkyl group, the alkylamine group, the N-arylalkylamine group, the alkylthioxy group, the alkylsulfoxy group, and the N-alkylheteroarylamine group is the same as the above-described examples of the alkyl group. Specifically, examples of the alkylthioxy group include a methylthioxy group, an ethylthioxy group, a tert-butylthioxy group, a hexylthioxy group, an octylthioxy group, and the like, and examples of the alkylsulfoxy group include mesyl, an ethylsulfoxy group, a propylsulfoxy group, a butylsulfoxy group, and the like, but the examples are not limited thereto.

In the present specification, the alkenyl group can be straight-chained or branched, and the number of carbon atoms thereof is not particularly limited, but is preferably 2 to 30. Specific examples thereof include vinyl, 1-propenyl, isopropenyl, 1-butenyl, 2-butenyl, 3-butenyl, 1-pentenyl, 2-pentenyl, 3-pentenyl, 3-methyl-l-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, an arylalkenyl group means an alkenyl group substituted with an aryl group.

In the present specification, specific examples of a silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group can be —BR₁₀₀R₁₀₁, and R₁₀₀ and R₁₀₁ are the same as or different from each other, and can be each independently selected from the group consisting of hydrogen, deuterium, halogen, a nitrile group, a substituted or unsubstituted monocyclic or polycyclic cycloalkyl group having 3 to 30 carbon atoms, a substituted or unsubstituted straight-chained or branched alkyl group having 1 to 30 carbon atoms, a substituted or unsubstituted monocyclic or polycyclic aryl group having 6 to 30 carbon atoms, and a substituted or unsubstituted monocyclic or polycyclic heteroaryl group having 2 to 30 carbon atoms.

In the present specification, specific examples of a phosphine oxide group include a diphenylphosphine oxide group, dinaphthylphosphine oxide group, and the like, but are not limited thereto.

In the present specification, an aryl group is not particularly limited, but has preferably 6 to 30 carbon atoms, and the aryl group can be monocyclic or polycyclic.

When the aryl group is a monocyclic aryl group, the number of carbon atoms thereof is not particularly limited, but is preferably 6 to 30. Specific examples of the monocyclic aryl group include a phenyl group, a biphenyl group, a terphenyl group, and the like, but are not limited thereto.

When the aryl group is a polycyclic aryl group, the number of carbon atoms thereof is not particularly limited, but is preferably 10 to 30. Specific examples of the polycyclic aryl group include a naphthyl group, an anthracenyl group, a phenanthryl group, a triphenylene group, a pyrenyl group, a phenalenyl group, a perylenyl group, a chrysenyl group, a fluorenyl group, and the like, but are not limited thereto.

In the present specification, the fluorenyl group can be substituted, and adjacent substituents can be bonded to each other to form a ring.

When the fluorenyl group is substituted, the substituent can be

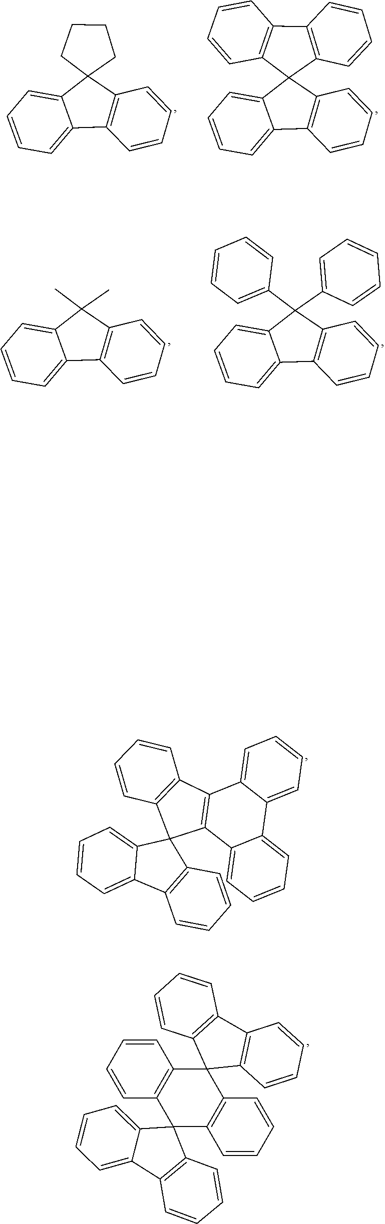

and the like. However, the substituent is not limited thereto.

In the present specification, the “adjacent” group can mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring can be interpreted as groups which are “adjacent” to each other.

In the present specification, the aryl group in the arylalkyl group, the arylalkenyl group, the aryloxy group, the arylthioxy group, the arylsulfoxy group, the N-arylalkylamine group, the N-arylheteroarylamine group, and the arylphosphine group is the same as the above-described examples of the aryl group. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, examples of the arylthioxy group include a phenylthioxy group, a 2-methylphenylthioxy group, a 4-tert-butylphenylthioxy group, and the like, and examples of the arylsulfoxy group include a benzenesulfoxy group, a p-toluenesulfoxy group, and the like, but the examples are not limited thereto.

In the present specification, examples of an arylamine group include a substituted or unsubstituted monoarylamine group, a substituted or unsubstituted diarylamine group, or a substituted or unsubstituted triarylamine group. The aryl group in the arylamine group can be a monocyclic aryl group or a polycyclic aryl group. The arylamine group including two or more aryl groups can include a monocyclic aryl group, a polycyclic aryl group, or both a monocyclic aryl group and a polycyclic aryl group. For example, the aryl group in the arylamine group can be selected from the above-described examples of the aryl group.

In the present specification, a heteroaryl group includes one or more atoms other than carbon, that is, one or more heteroatoms, and specifically, the heteroatom can include one or more atoms selected from the group consisting of O, N, Se, S, and the like. The number of carbon atoms thereof is not particularly limited, but is preferably 2 to 30, and the heteroaryl group can be monocyclic or polycyclic. Examples of the heterocyclic group include a thiophene group, a furanyl group, a pyrrole group, an imidazolyl group, a triazolyl group, an oxazolyl group, an oxadiazolyl group, a pyridyl group, a bipyridyl group, a pyrimidyl group, a triazinyl group, a triazolyl group, an acridyl group, a pyridazinyl group, a pyrazinyl group, a quinolinyl group, a quinazolinyl group, a quinoxalinyl group, a phthalazinyl group, a pyridopyrimidyl group, a pyridopyrazinyl group, a pyrazinopyrazinyl group, an isoquinolinyl group, an indolyl group, a carbazolyl group, a benzoxazolyl group, a benzimidazolyl group, a benzothiazolyl group, a benzocarbazolyl group, a benzothiophene group, a dibenzothiophene group, a benzofuranyl group, a phenanthrolinyl group (phenanthroline), a thiazolyl group, an isoxazolyl group, an oxadiazolyl group, a thiadiazolyl group, a benzothiazolyl group, a phenothiazinyl group, a phenoxazinyl group, a dibenzofuranyl group, and the like, but are not limited thereto.

In the present specification, examples of the heteroarylamine group include a substituted or unsubstituted monoheteroarylamine group, a substituted or unsubstituted diheteroarylamine group, or a substituted or unsubstituted triheteroarylamine group. The heteroarylamine group including two or more heteroaryl groups can include a monocyclic heteroaryl group, a polycyclic heteroaryl group, or both a monocyclic heteroaryl group and a polycyclic heteroaryl group. For example, the heteroaryl group in the heteroarylamine group can be selected from the above-described examples of the heteroaryl group.

In the present specification, examples of the heteroaryl group in the N-arylheteroarylamine group and the N-alkylheteroarylamine group are the same as the above-described examples of the heteroaryl group.

In the present specification, an arylene group means a group having two bonding positions in an aryl group, that is, a divalent group. The above-described description on the aryl group can be applied to the arylene group, except that the arylene groups are each a divalent group.

According to an exemplary embodiment of the present specification, provided is an organic light emitting device including: a first electrode; a second electrode; and an organic material layer having one or more layers provided between the first electrode and the second electrode, in which the organic material layer includes a light emitting layer, and the light emitting layer includes the compound of Chemical Formula 1 and the compound of Chemical Formula 2. In this case, the first electrode and the second electrode can be provided to face each other.

According to an exemplary embodiment of the present specification, the compound of Chemical Formula 1 is the following Chemical Formula 1-1:

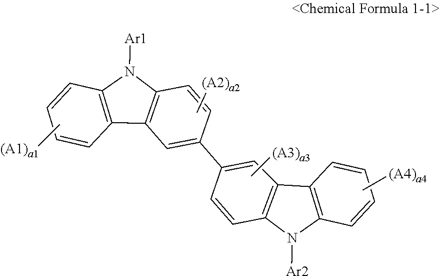

wherein in Chemical Formula 1-1, Ar1, Ar2, A1 to A4, and al to a4 are the same as definitions in Chemical Formula 1.

According to an exemplary embodiment of the present specification, the compound of Chemical Formula 1 is any one of the following Chemical Formulae 1-1-1 to 1-1-3:

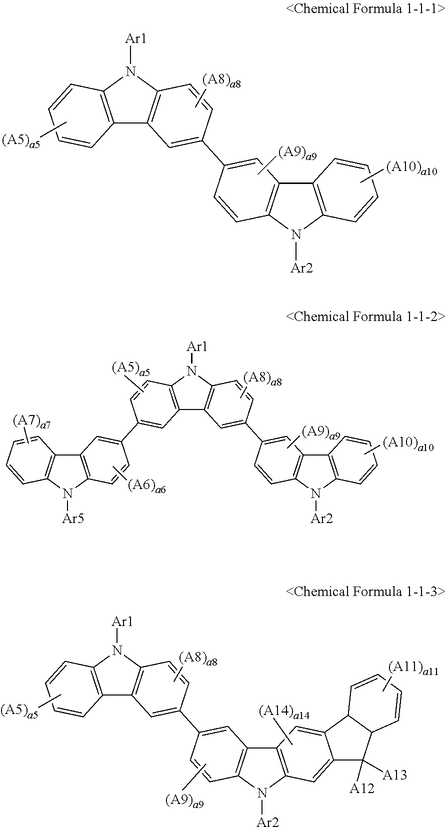

wherein in Chemical Formulae 1-1-1 to 1-1-3:

Ar1, Ar2, and Ar5 are the same as or different from each other, and are each independently a substituted or unsubstituted aryl group having 6 to 20 carbon atoms;

A5 to A11 and A14 are the same as or different from each other, and are each independently hydrogen, deuterium, or a substituted or unsubstituted aryl group;

A12 and A13 are the same as or different from each other, and are each independently a substituted or unsubstituted alkyl group or a substituted or unsubstituted aryl group;

a5, a6, a8, and a9 are each an integer from 1 to 3, and a7, a10, and all are each an integer from 1 to 4;

when a5 to all are each an integer of 2 or higher, two or more structures in the parenthesis are the same as or different from each other; and

a14 is 1 or 2, and when a14 is 2, structures in the parenthesis are the same as or different from each other.

According to an exemplary embodiment of the present specification, Ar1 and Ar2 are the same as or different from each other, and are each independently a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group.

According to an exemplary embodiment of the present specification, Ar1 and Ar2 are the same as or different from each other, and are each independently a phenyl group, a biphenyl group, a terphenyl group, a naphthyl group, or a phenanthrenyl group.

According to an exemplary embodiment of the present specification, A1 to A4 are the same as or different from each other, and are each independently hydrogen, deuterium, a substituted or unsubstituted aryl group, or a substituted or unsubstituted carbazole group, or are bonded to an adjacent substituent to form a substituted or unsubstituted hydrocarbon ring.

According to an exemplary embodiment of the present specification, A1 to A4 are the same as or different from each other, and are each independently hydrogen or deuterium.

According to an exemplary embodiment of the present specification, A1 to A4 are the same as or different from each other, and are each independently hydrogen.

According to an exemplary embodiment of the present specification, A5 to A11 and A14 are the same as or different from each other, and are each independently hydrogen, deuterium, or a substituted or unsubstituted aryl group.

According to an exemplary embodiment of the present specification, A5 to A11 and A14 are the same as or different from each other, and are each independently hydrogen or deuterium.

According to an exemplary embodiment of the present specification, A5 to A11 and A14 are the same as or different from each other, and are each independently hydrogen.

According to an exemplary embodiment of the present specification, A12 and A13 are the same as or different from each other, and are each independently a substituted or unsubstituted alkyl group or a substituted or unsubstituted aryl group.

According to an exemplary embodiment of the present specification, A12 and A13 are the same as or different from each other, and are each independently a substituted or unsubstituted alkyl group.

According to an exemplary embodiment of the present specification, A12 and A13 are the same as or different from each other, and are each independently an alkyl group.

According to an exemplary embodiment of the present specification, A12 and A13 are a methyl group.

According to an exemplary embodiment of the present specification, Ar5 is a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group.

According to an exemplary embodiment of the present specification, Ar5 is a phenyl group, a biphenyl group, a terphenyl group, a naphthyl group, or a phenanthrenyl group.

According to an exemplary embodiment of the present specification, Ar5 is a phenyl group.

According to an exemplary embodiment of the present specification, Chemical Formula 1 is selected from the following compounds:

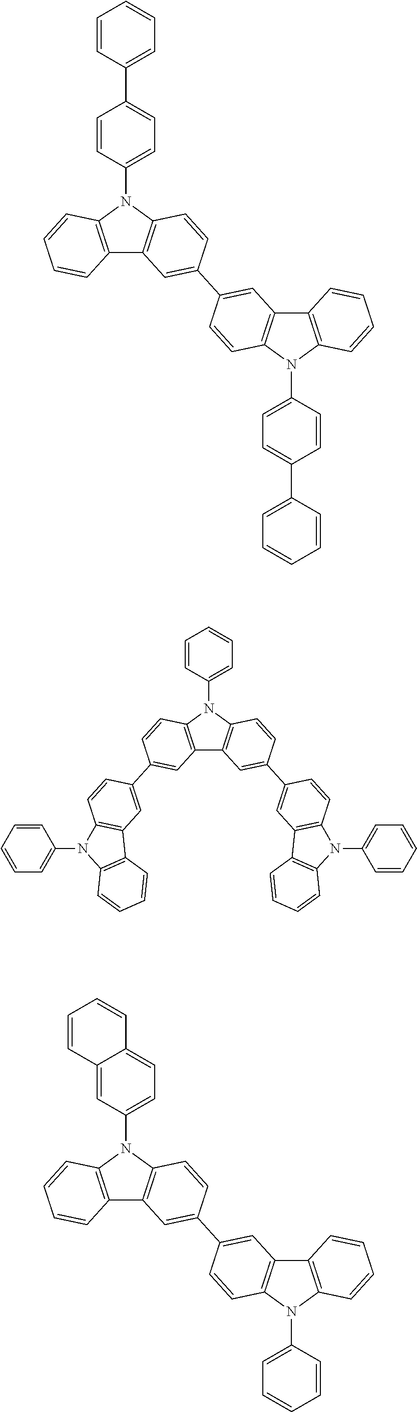 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccc5ccccc5c4)ccc32)cc1]

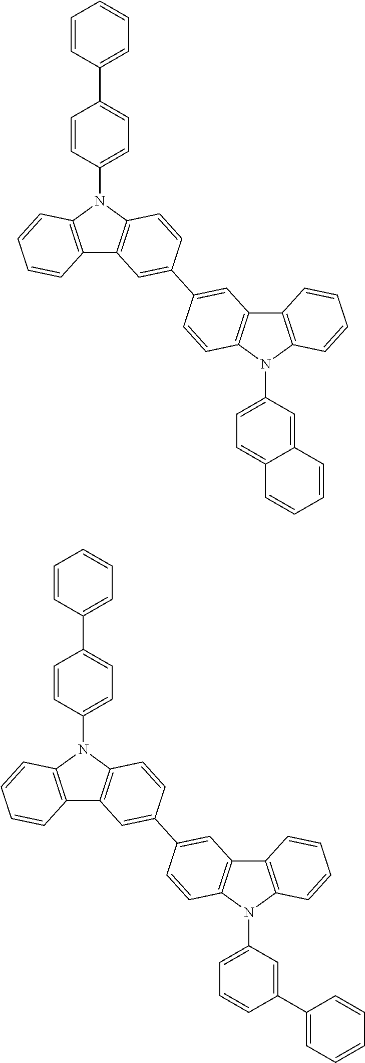 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

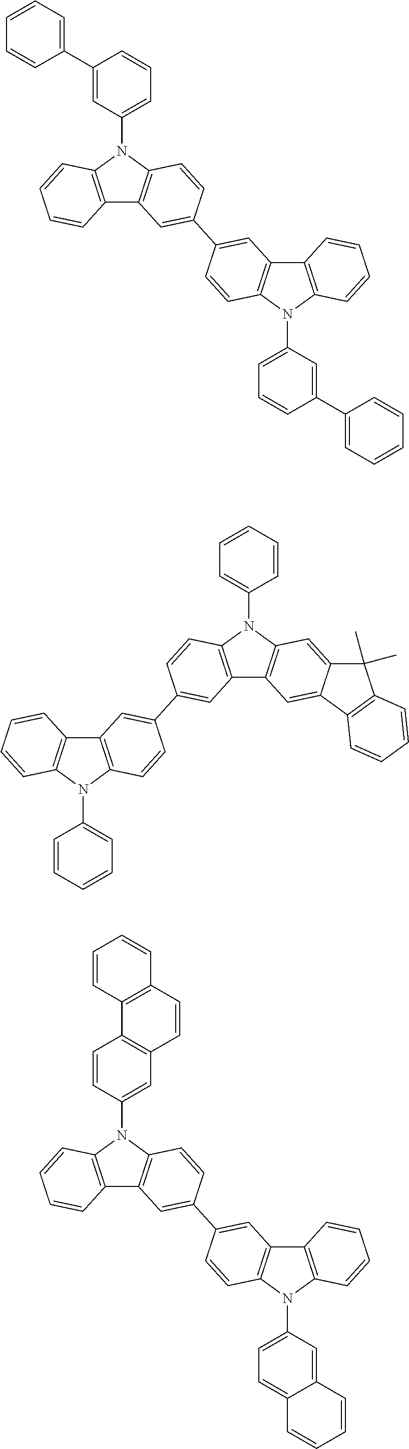 [SMILES: c1ccc(-c2cccc(-n3c4ccccc4c4cc(-c5ccc6c(c5)c5ccccc5n6-c5cccc(-c6ccccc6)c5)ccc43)c2)cc1 ; CC1(C)c2ccccc2-c2cc3c4cc(-c5ccc6c(c5)c5ccccc5n6-c5ccccc5)ccc4n(-c4ccccc4)c3cc21 ; c1ccc2cc(-n3c4ccccc4c4cc(-c5ccc6c(c5)c5ccccc5n6-c5ccc6c(ccc7ccccc76)c5)ccc43)ccc2c1]

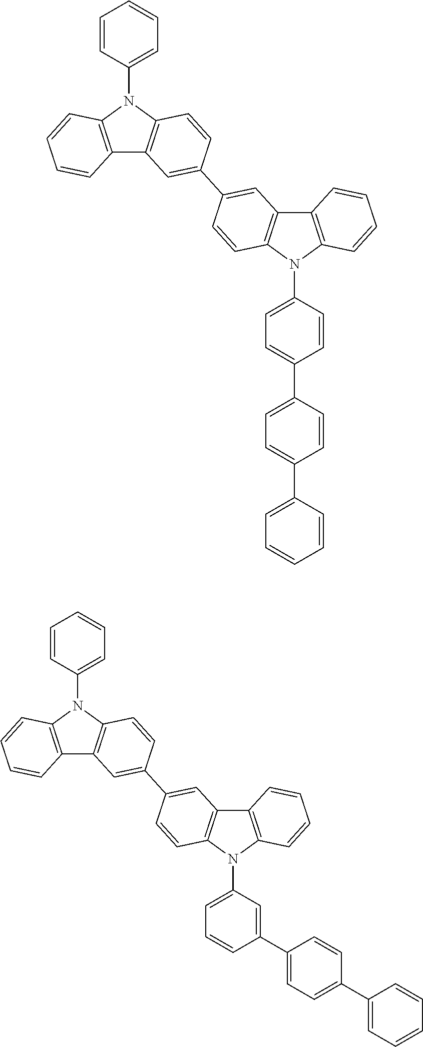 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

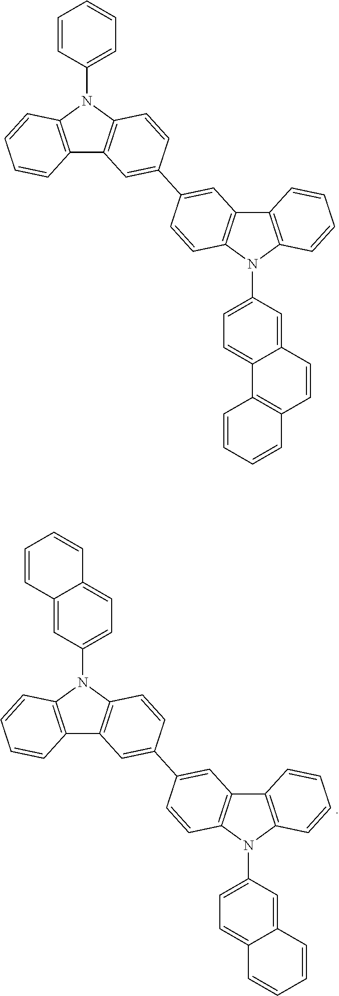 [SMILES: c1ccc(-n2c3ccccc3c3cc(-c4ccc5c(c4)c4ccccc4n5-c4ccc5c(ccc6ccccc65)c4)ccc32)cc1 ; c1ccc2cc(-n3c4ccccc4c4cc(-c5ccc6c(c5)c5ccccc5n6-c5ccc6ccccc6c5)ccc43)ccc2c1]

According to an exemplary embodiment of the present specification, the compound of Chemical Formula 2 is the following Chemical Formula 2-1:

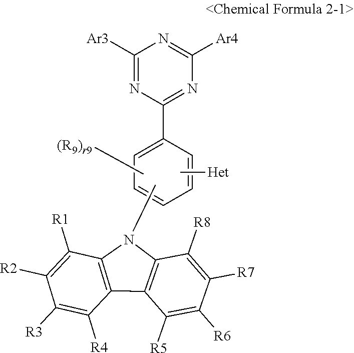

wherein in Chemical Formula 2-1:

Ar3, Ar4, and Het are the same as the definitions in Chemical Formula 2;

R1 to R9 are the same as or different from each other, and are each independently hydrogen, deuterium, or a substituted or unsubstituted aryl group; and

r9 is an integer from 1 to 3, and when r9 is an integer of 2 or higher, two or more structures in the parenthesis are the same as or different from each other.

According to an exemplary embodiment of the present specification, the compound of Chemical Formula 2 is the following Chemical Formula 2-2:

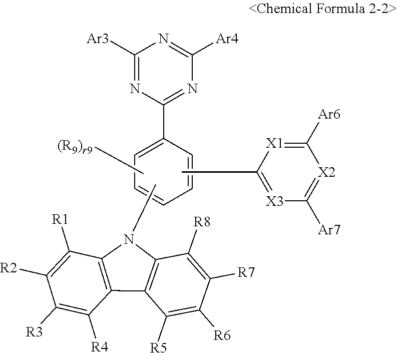

wherein in Chemical Formula 2-2:

Ar3, Ar4, Ar6, and Ar7 are the same as or different from each other, and are each independently a substituted or unsubstituted aryl group;

at least one of X1 to X3 is N, and the others are CH;

R1 to R9 are the same as or different from each other, and are each independently hydrogen, deuterium, or a substituted or unsubstituted aryl group; and

r9 is an integer from 1 to 3, and when r9 is an integer of 2 or higher, two or more structures in the parenthesis are the same as or different from each other.

According to an exemplary embodiment of the present specification, the compound of Chemical Formula 2 is any one of the following Chemical Formulae 2-2-1 to 2-2-3:

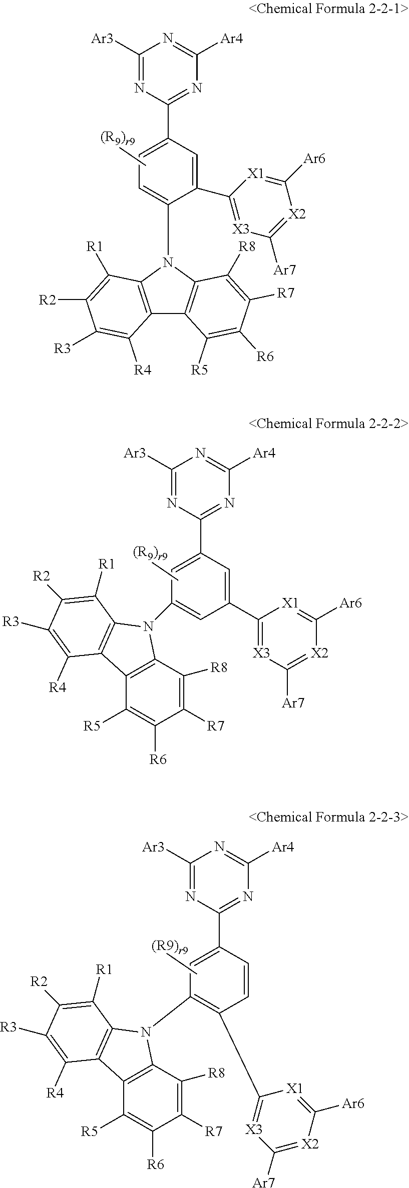

wherein in Chemical Formulae 2-2-1 to 2-2-3:

Ar3, Ar4, Ar6, and Ar7 are the same as or different from each other, and are each independently a substituted or unsubstituted aryl group;

at least one of X1 to X3 is N, and the others are CH;

R1 to R9 are the same as or different from each other, and are each independently hydrogen, deuterium, or a substituted or unsubstituted aryl group; and

r9 is an integer from 1 to 3, and when r9 is an integer of 2 or higher, two or more structures in the parenthesis are the same as or different from each other.

According to an exemplary embodiment of the present specification, at least two of X1 to X3 are N, and the other is CH.

According to an exemplary embodiment of the present specification, X1 to X3 are N.

According to an exemplary embodiment of the present specification, Ar3, Ar4, Ar6, and Ar7 are the same as or different from each other, and are each independently a substituted or unsubstituted aryl group having 6 to 20 carbon atoms.

According to an exemplary embodiment of the present specification, Ar3, Ar4, Ar6, and Ar7 are the same as or different from each other, and are each independently an aryl group having 6 to 20 carbon atoms.

According to an exemplary embodiment of the present specification, Ar3, Ar4, Ar6, and Ar7 are a phenyl group.

According to an exemplary embodiment of the present specification, R1 to R8 are the same as or different from each other, and are each independently hydrogen, deuterium; or an aryl group.

According to an exemplary embodiment of the present specification, R1 to R8 are the same as or different from each other, and are each independently hydrogen, deuterium, or a phenyl group.

According to an exemplary embodiment of the present specification, R9 is hydrogen or deuterium.

According to an exemplary embodiment of the present specification, R9 is hydrogen.

According to an exemplary embodiment of the present specification, Chemical Formula 2 is selected from the following compounds:

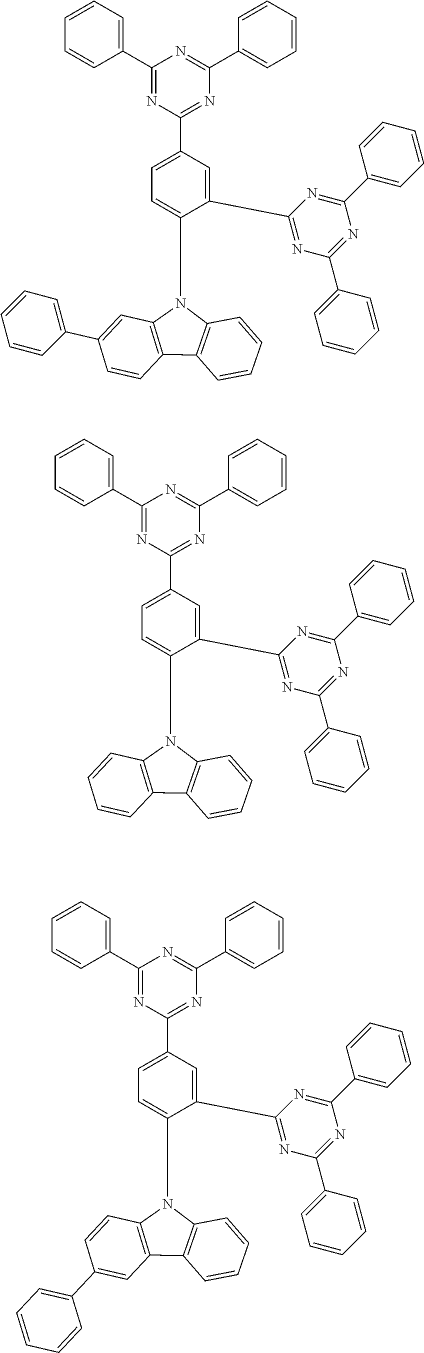 [SMILES: c1ccc(-c2ccc3c4ccccc4n(-c4ccc(-c5nc(-c6ccccc6)nc(-c6ccccc6)n5)cc4-c4nc(-c5ccccc5)nc(-c5ccccc5)n4)c3c2)cc1 ; c1ccc(-c2nc(-c3ccccc3)nc(-c3ccc(-n4c5ccccc5c5ccccc54)c(-c4nc(-c5ccccc5)nc(-c5ccccc5)n4)c3)n2)cc1 ; c1ccc(-c2ccc3c(c2)c2ccccc2n3-c2ccc(-c3nc(-c4ccccc4)nc(-c4ccccc4)n3)cc2-c2nc(-c3ccccc3)nc(-c3ccccc3)n2)cc1]

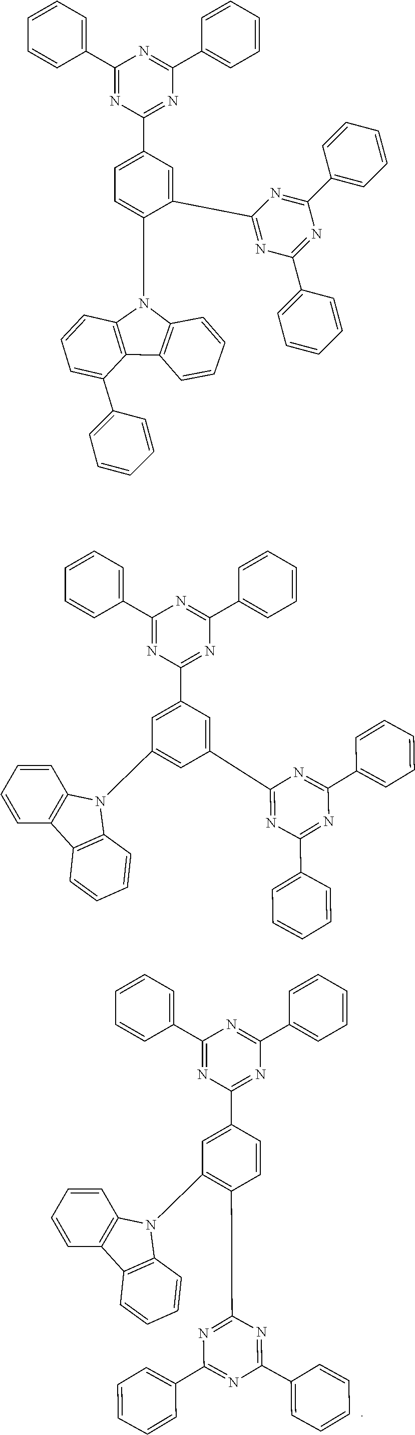

According to an exemplary embodiment of the present specification, the organic material layer of the organic light emitting device of the present specification can be composed of a mono-layer structure, but can be composed of a multi-layer structure in which two or more organic material layers are stacked. For example, the organic light emitting device of the present invention can have a structure including a hole injection layer, a hole transport layer, an electron blocking layer, a light emitting layer, a hole blocking layer, an electron transport layer, an electron injection layer, and the like as organic material layers. However, the structure of the organic light emitting device is not limited thereto, and can include fewer or more organic layers.

The organic light emitting device can have, for example, a stacking structure described below, but the stacking structure is not limited thereto.

(1) Positive electrode/Hole transport layer/Light emitting layer/Negative electrode

(2) Positive electrode/Hole injection layer/Hole transport layer/Light emitting layer/Negative electrode

(3) Positive electrode/Hole transport layer/Light emitting layer/Electron transport layer/Negative electrode

(4) Positive electrode/Hole transport layer/Light emitting layer/Electron transport layer/Electron injection layer/Negative electrode

(5) Positive electrode/Hole injection layer/Hole transport layer/Light emitting layer/Electron transport layer/Negative electrode

(6) Positive electrode/Hole injection layer/Hole transport layer/Light emitting layer/Electron transport layer/Electron injection layer/Negative electrode

(7) Positive electrode/Hole transport layer/Electron blocking layer/Light emitting layer/Electron transport layer/Negative electrode

(8) Positive electrode/ Hole transport layer/Electron blocking layer/Light emitting layer/Electron transport layer/Electron injection layer/Negative electrode

(9) Positive electrode/Hole injection layer/Hole transport layer/Electron blocking layer/Light emitting layer/Electron transport layer/Negative electrode

(10) Positive electrode/Hole injection layer/Hole transport layer/Electron blocking layer/Light emitting layer/Electron transport layer/Electron injection layer/Negative electrode

(11) Positive electrode/Hole transport layer/Light emitting layer/Hole blocking layer/Electron transport layer/Negative electrode

(12) Positive electrode/Hole transport layer/Light emitting layer/Hole blocking layer/Electron transport layer/Electron injection layer/Negative electrode

(13) Positive electrode/Hole injection layer/Hole transport layer/Light emitting layer/Hole blocking layer/Electron transport layer/Negative electrode

(14) Positive electrode/Hole injection layer/Hole transport layer/Light emitting layer/Hole blocking layer/Electron transport layer/Electron injection layer/Negative electrode

(15) Positive electrode/Hole injection layer/Hole transport layer/Electron blocking layer/Light emitting layer/Hole blocking layer/Electron transport layer/Electron injection layer/Negative electrode

(16) Positive electrode/ Hole injection layer/Hole transport layer/Electron blocking layer/Light emitting layer/Hole blocking layer/Electron transport and injection layer/Negative electrode

(17) Positive electrode/Hole injection layer/First hole transport layer/Second hole transport layer/Electron blocking layer/Light emitting layer/Hole blocking layer/Electron transport and injection layer/Negative electrode

(18) Positive electrode/Hole injection layer/First hole transport layer/Second hole transport layer/Electron blocking layer/Light emitting layer/Hole blocking layer/Electron transport layer/Electron injection layer/Negative electrode

(19) Positive electrode/Hole injection layer/First hole transport layer/Second hole transport layer/Electron blocking layer/Light emitting layer/Hole blocking layer/Electron transport layer/Electron injection layer/Negative electrode/Capping layer

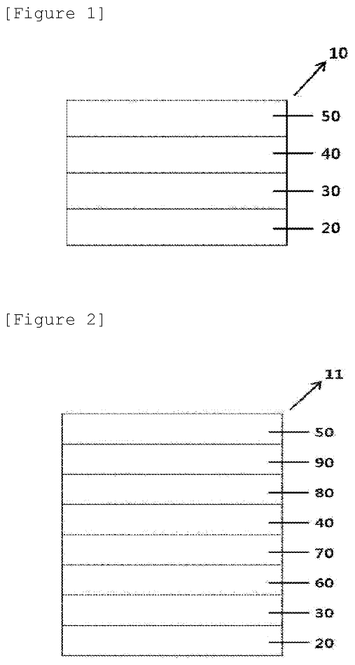

For example, the structure of the organic light emitting device of the present specification can be a structure illustrated in FIGS. 1 and 2, but is not limited thereto.

FIG. 1 exemplifies a structure of an organic light emitting device 10 in which a first electrode 30, a light emitting layer 40, and a second electrode 50 are sequentially stacked on a substrate 20. FIG. 1 is an exemplified structure of the organic light emitting device according to an exemplary embodiment of the present specification, and can further include other organic material layers.

FIG. 2 exemplifies a structure of an organic light emitting device 11 in which the first electrode 30, a hole injection layer 60, a hole transport layer 70, the light emitting layer 40, an electron transport layer 80, an electron injection layer 90, and the second electrode 50 are sequentially stacked on the substrate 20. FIG. 2 is an exemplified structure according to an exemplary embodiment of the present specification, and the structure can further include other organic material layers.

According to an exemplary embodiment of the present specification, the light emitting layer includes a host and a dopant, and the host includes the compound of Chemical Formula 1 and the compound of Chemical Formula 2.

In the light emitting layer, a weight ratio of the compound of Chemical Formula 1 and the compound of Chemical Formula 2 is preferably 99:1 to 1:99, or 95:5 to 5:95.

In an exemplary embodiment of the present specification, the light emitting layer includes the compounds of Chemical Formulae 1 and 2 as a host, and can include other organic compounds, metals, or metal compounds as a dopant.

According to an exemplary embodiment of the present specification, the organic material layer can further include one or more layers selected from the group consisting of a hole injection layer, a hole transport layer, an electron blocking layer, a light emitting layer, a hole blocking layer, an electron transport layer, and an electron injection layer.

The organic light emitting device of the present specification can be manufactured by materials and methods known in the art, except that the light emitting layer includes the heterocyclic compounds of Chemical Formulae 1 and 2.

When the organic light emitting device includes a plurality of organic material layers, the organic material layers can be formed of the same material or different materials.

For example, the organic light emitting device of the present specification can be manufactured by sequentially stacking a first electrode, an organic material layer, and a second electrode on a substrate. In this case, the organic light emitting device can be manufactured by depositing a metal or a metal oxide having conductivity, or an alloy thereof on a substrate to form a first electrode, forming an organic material layer including a hole injection layer, a hole transport layer, a light emitting layer, and an electron transport layer thereon, and then depositing a material, which can be used as a second electrode, thereon, by using a physical vapor deposition (PVD) method such as sputtering or e-beam evaporation. In addition to the method described above, an organic light emitting device can be made by sequentially depositing a second electrode material, an organic material layer, and a first electrode material on a substrate.

Further, the compounds of Chemical Formulae 1 and 2 can be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

According to an exemplary embodiment of the present specification, the first electrode is a positive electrode, and the second electrode is a negative electrode.

According to another exemplary embodiment of the present specification, the first electrode is a negative electrode, and the second electrode is a positive electrode.

As the positive electrode material, materials having a high work function are usually preferred so as to facilitate the injection of holes into an organic material layer. Specific examples of the positive electrode material which can be used in the present invention include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SNO2:Sb; a conductive polymer such as poly(3-methylthiophene), poly[3,4-(ethylene-1,2-dioxy)thiophene] (PEDOT), polypyrrole, and polyaniline; and the like, but are not limited thereto.

As the negative electrode material, materials having a low work function are usually preferred so as to facilitate the injection of electrons into an organic material layer. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material such as LiF/Al or LiO₂/Al and Mg/Ag; and the like, but are not limited thereto.

The hole injection layer is a layer which injects holes from an electrode, and a hole injection material is preferably a compound which has a capability of transporting holes and thus has an effect of injecting holes at a positive electrode and an excellent effect of injecting holes into a light emitting layer or a light emitting material, prevents excitons produced from the light emitting layer from moving to an electron injection layer or an electron injection material, and is also excellent in the ability to form a thin film. The highest occupied molecular orbital (HOMO) of the hole injection material is preferably a value between the work function of the positive electrode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based electrically conductive polymers, and the like, but are not limited thereto.

The hole transport layer is a layer which accepts holes from a hole injection layer and transports the holes to a light emitting layer, and a hole transport material is suitably a material having high hole mobility which can accept holes from a positive electrode or a hole injection layer and transfer the holes to a light emitting layer. Specific examples thereof include arylamine-based organic materials, conductive polymers, block copolymers having both conjugated portions and non-conjugated portions, and the like, but are not limited thereto.

The electron blocking layer is a layer which can improve the service life and efficiency of the device by preventing electrons injected from an electron injection layer from passing through a light emitting layer and entering a hole injection layer, and can be formed at an appropriate portion between the light emitting layer and the hole injection layer by using publicly-known materials which are rich in electrons, if necessary. A material for the electron blocking layer needs to be selected in consideration of the characteristics of a light emitting layer adjacent to the entire device structure, and is often selected from materials that can be usually used as a material for the hole transport layer.

According to an exemplary embodiment of the present specification, it is preferred to select a compound of the following EBL1 for the electron blocking layer adjacent to the light emitting layer including the compound of Chemical Formula 1 and the compound of Chemical Formula 2. An electron transport layer material in which electrons were well charged so as to match the material for a light emitting layer was used to make the light emitting layer rich in electrons, so that the effect of the invention of increasing the light emitting efficiency was shown, but there occurred a problem in that electrons rich in the light emitting layer entered the hole transport layer, and thus made the hole transport layer deteriorate. Thus, the electron blocking layer is indispensable in the device structure of the present specification, and the following EBL1 which is a material rich in holes is used to prevent electrons flowing from the light emitting layer, so that it was possible to achieve long service life and high efficiency by preventing the hole transport layer from deteriorating.

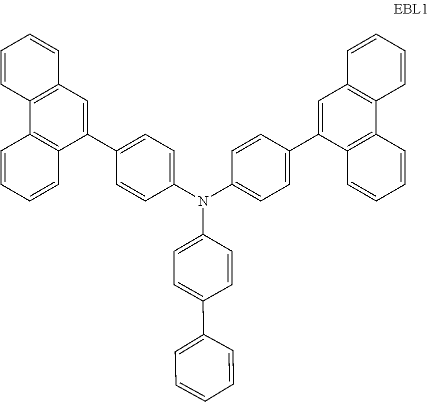 [SMILES: c1ccc(-c2ccc(N(c3ccc(-c4cc5ccccc5c5ccccc45)cc3)c3ccc(-c4cc5ccccc5c5ccccc45)cc3)cc2)cc1]

A light emitting material for the light emitting layer is a material which can emit light in a visible light region by accepting and combining holes and electrons from a hole transport layer and an electron transport layer, respectively, and is preferably a material having high quantum efficiency for fluorescence or phosphorescence. In the present specification, the compounds of Chemical Formulae 1 and 2 are included, and another additional light emitting material can be further included. Specific examples thereof include: 8-hydroxy-quinoline aluminum complexes (Alq₃); carbazole-based compounds; dimerized styryl compounds; BAlq; 10-hydroxybenzoquinoline-metal compounds; benzoxazole-based, benzothiazole-based and benzimidazole-based compounds; poly(p-phenylenevinylene) (PPV)-based polymers; spiro compounds; polyfluorene, rubrene, and the like, but are not limited thereto.

The light emitting layer can include a host material and a dopant material. The host material includes the compound of Chemical Formulae 1 and 2, and can further include another additional light emitting material. Examples of the additional light emitting material include a fused aromatic ring derivative, or a hetero ring-containing compound, and the like. Specific examples of the fused aromatic ring derivative include an anthracene derivative, a pyrene derivative, a naphthalene derivative, a pentacene derivative, a phenanthrene compound, a fluoranthene compound, and the like, and examples of the hetero ring-containing compound include a carbazole derivative, a dibenzofuran derivative, a ladder-type furan compound, a pyrimidine derivative, and the like, but the examples thereof are not limited thereto.

Examples of the dopant material include an aromatic amine derivative, a styrylamine compound, a boron complex, a fluoranthene compound, a metal complex, and the like. Specifically, the aromatic amine derivative is a fused aromatic ring derivative having a substituted or unsubstituted arylamino group, and examples thereof include a pyrene, an anthracene, a chrysene, a periflanthene, and the like, which have an arylamino group, and the styrylamine compound is a compound in which a substituted or unsubstituted arylamine is substituted with at least one arylvinyl group, and one or two or more substituents selected from the group consisting of an aryl group, a silyl group, an alkyl group, a cycloalkyl group, and an arylamino group is or are substituted or unsubstituted. Specific examples thereof include styrylamine, styryldiamine, styryltriamine, styryltetramine, and the like, but are not limited thereto. Further, examples of the metal complex include iridium complexes, platinum complexes, and the like, but are not limited thereto.

According to an exemplary embodiment of the present specification, the light emitting layer further includes a phosphorescent dopant.

According to an exemplary embodiment of the present specification, the phosphorescent dopant can be a metal complex.

According to an exemplary embodiment of the present specification, examples of the phosphorescent dopant include an iridium complex, a platinum complex, and the like, but are not limited thereto.

According to an exemplary embodiment of the present specification, the phosphorescent dopant can be any one of the following compounds, but is not limited thereto.

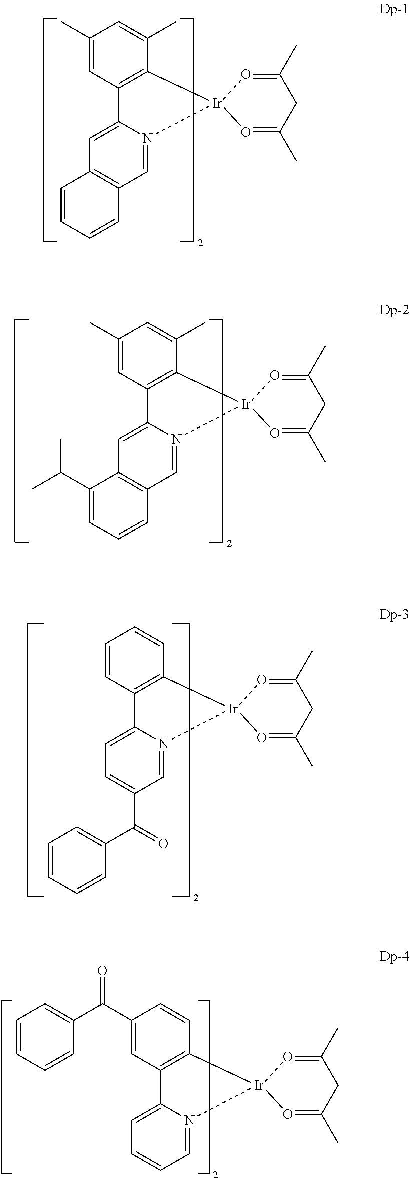

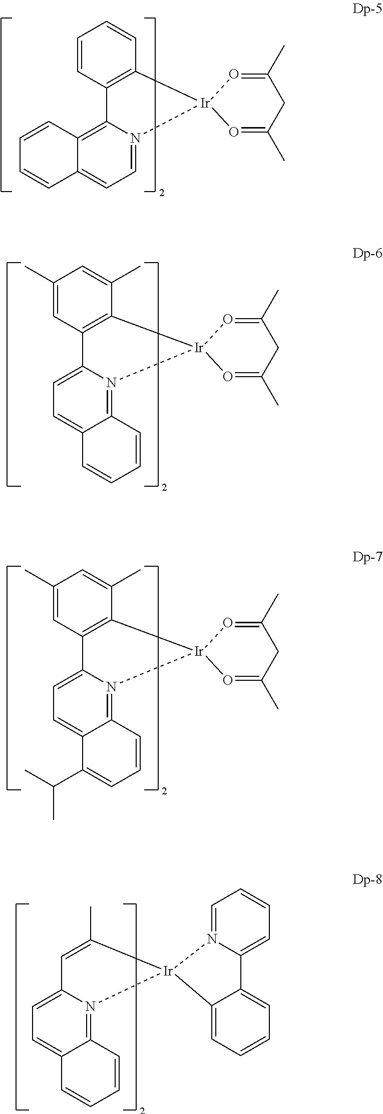

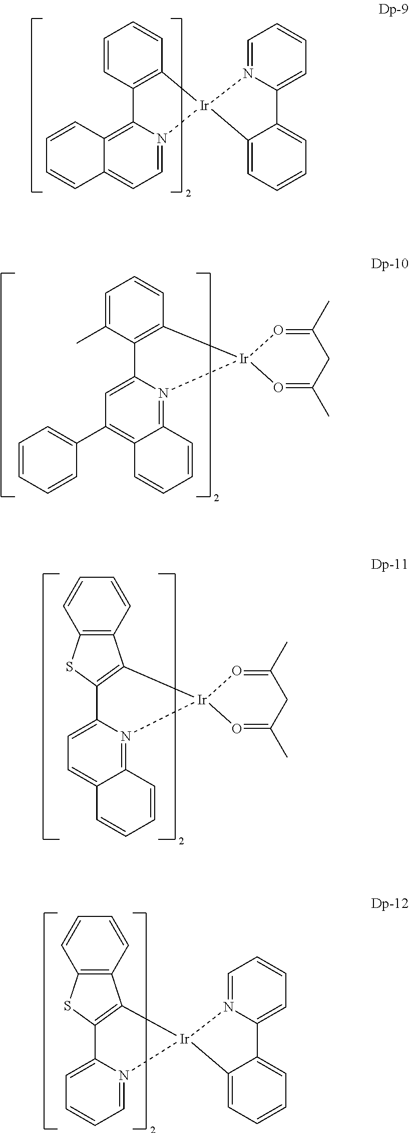

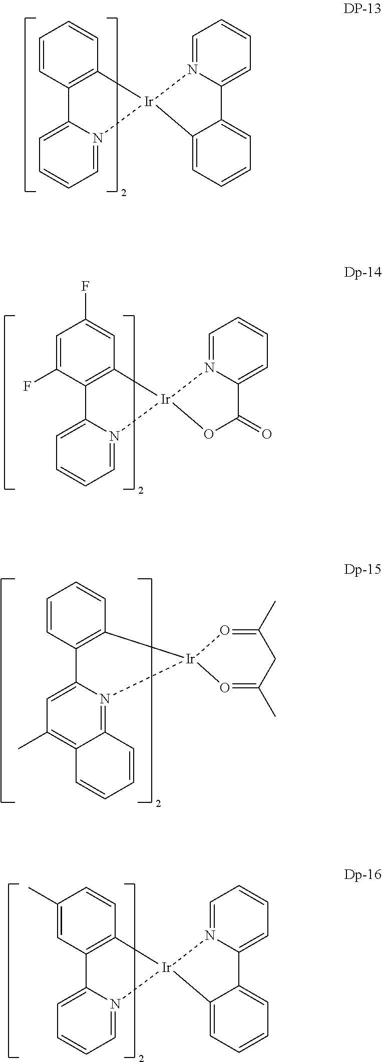

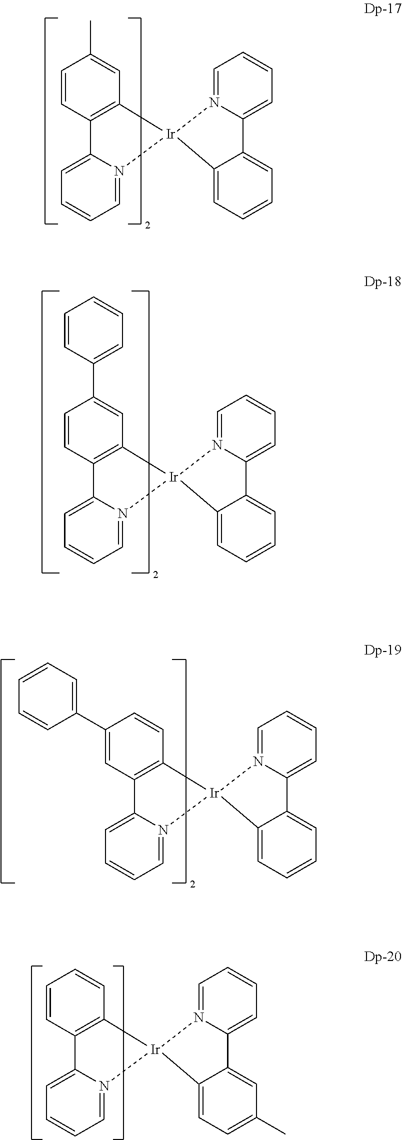

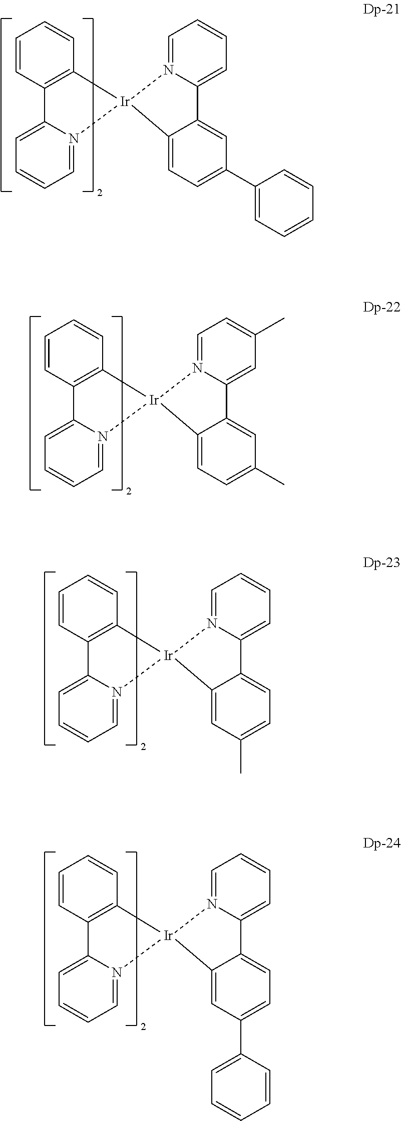

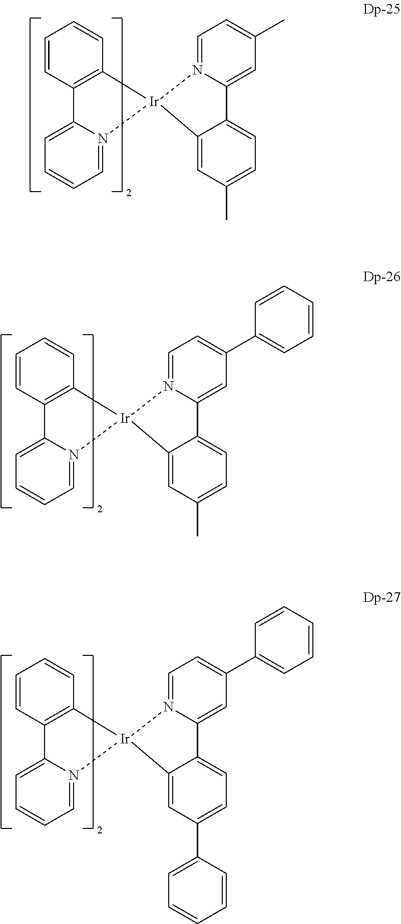

The hole blocking layer is a layer which can improve the service life and efficiency of the device by preventing holes injected from a hole injection layer from passing through a light emitting layer and entering an electron injection layer, and can be formed at an appropriate portion between the light emitting layer and the electron injection layer by using publicly-known materials, if necessary.

The electron transport layer is a layer which accepts electrons from an electron injection layer and transports the electrons to a light emitting layer, and an electron transport material is suitably a material having high electron mobility which can proficiently accept electrons from a negative electrode and transfer the electrons to a light emitting layer. Specific examples thereof include: Al complexes of 8-hydroxyquinoline; complexes including Alq₃; organic radical compounds; hydroxyflavone-metal complexes, and the like, but are not limited thereto. The electron transport layer can be used with any desired cathode material, as used according to the related art. In particular, examples of an appropriate cathode material include a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and an electron injection material is preferably a compound which has a capability of transporting electrons, an effect of injecting electrons from a negative electrode, and an excellent effect of injecting electrons into a light emitting layer or a light emitting material, prevents excitons produced from a light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

Examples of the metal complex compounds include 8-hydroxyquinolinato lithium, bis(8-hydroxyquinolinato) zinc, bis(8-hydroxyquinolinato) copper, bis(8-hydroxy-quinolinato) manganese, tris(8-hydroxyquinolinato) aluminum, tris(2-methyl-8-hydroxyquinolinato) aluminum, tris(8-hydroxyquinolinato) gallium, bis(10-hydroxy-benzo[h]quinolinato) beryllium, bis(10-hydroxybenzo[h]-quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

The capping layer is convexly deposited on a transparent cathode, and thus serves to collect the light emitted from the cathode. The electrical properties of the organic material forming the capping layer are not an item to be considered, but the refractive index of the formed thin film is important. The refractive index required for the capping layer can be 1.96 or less and 1.93 or more, and is preferably 1.94, under the wavelength condition of 520 nm.

The organic material forming the capping layer is not particularly limited, but can be selected, for example, from the following compounds:

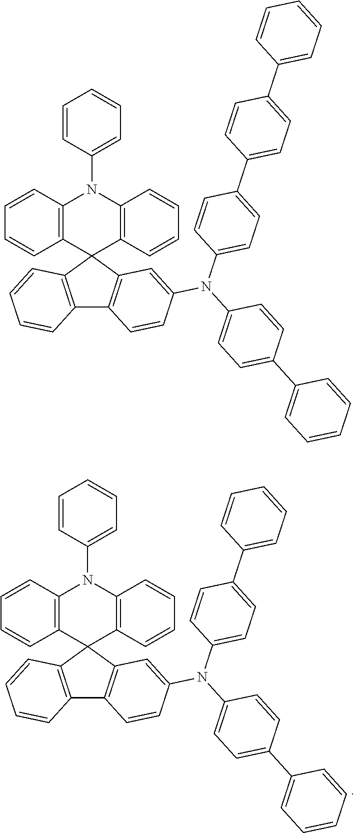 [SMILES: c1ccc(-c2ccc(-c3ccc(N(c4ccc(-c5ccccc5)cc4)c4ccc5c(c4)C4(c6ccccc6-5)c5ccccc5N(c5ccccc5)c5ccccc54)cc3)cc2)cc1 ; c1ccc(-c2ccc(N(c3ccc(-c4ccccc4)cc3)c3ccc4c(c3)C3(c5ccccc5-4)c4ccccc4N(c4ccccc4)c4ccccc43)cc2)cc1]

The organic light emitting device according to the present specification can be a top emission type, a bottom emission type, or a dual emission type according to the materials to be used.

According to an exemplary embodiment of the present specification, the compounds of Chemical Formulae 1 and 2 can be included in an organic solar cell or an organic transistor in addition to an organic light emitting device.

### Examples

Hereinafter, the present specification will be described in detail with reference to Examples for specifically describing the present specification. However, the Examples according to the present specification can be modified in various forms, and it is not interpreted that the scope of the present specification is limited to the Examples described below in detail. The Examples of the present specification are provided to explain the present specification more completely to a person with ordinary skill in the art.

### Synthesis Example 1

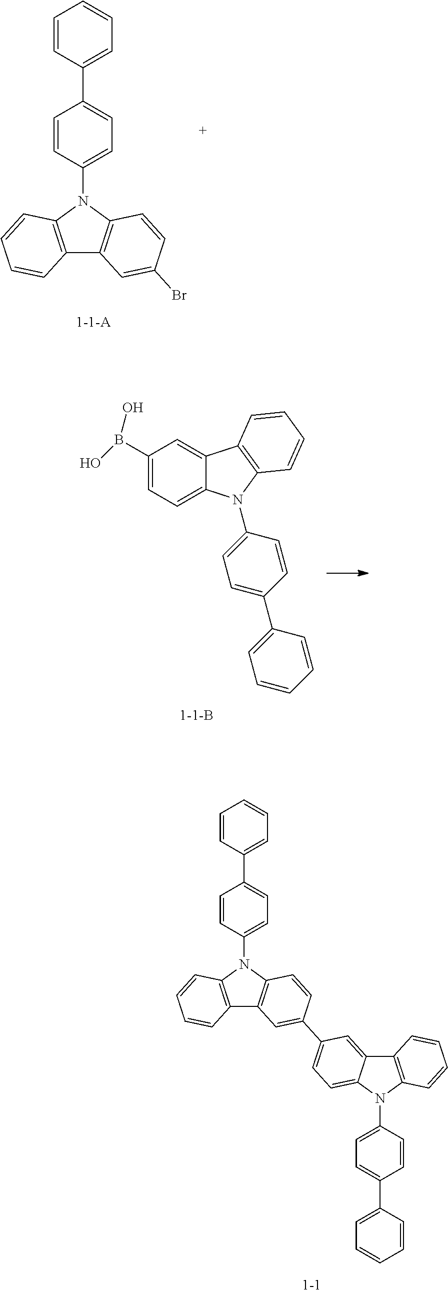 [SMILES: Brc1ccc2c(c1)c1ccccc1n2-c1ccc(-c2ccccc2)cc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

Compound 1-1-A (46.6 mmol), Compound 1-1-B (51.3 mmol), potassium carbonate (139.8 mmol), and tetrakis(triphenylphosphine)palladium(0) (0.9 mmol) were put into tetrahydrofuran, and the resulting mixture was refluxed for 12 hours. After completion of the reaction, the aqueous layer was removed, the organic layer was dried over magnesium sulfate, and then the filtered liquid was distilled under reduced pressure. After the obtained solid was dissolved in chloroform (500 ml), ethyl acetate (100 ml) was added thereto, the resulting mixture was recrystallized and then dried to obtain Compound 1-1 (yield 86%).

### Synthesis Example 2

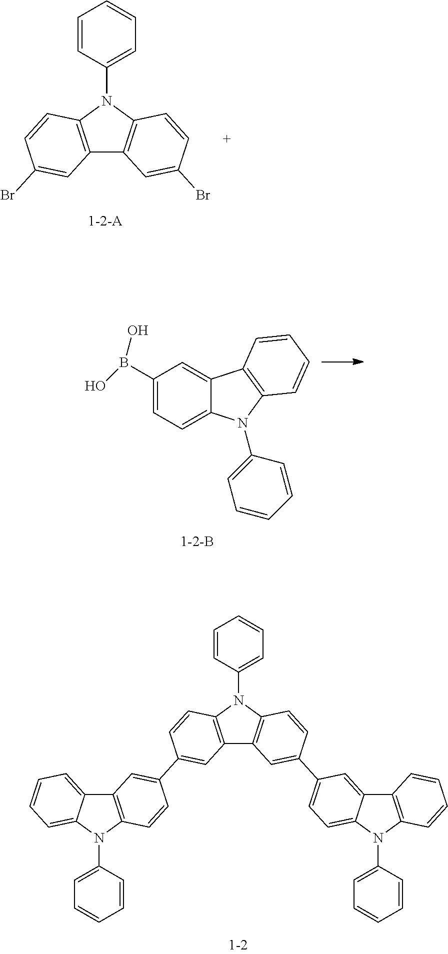 [SMILES: Brc1ccc2c(c1)c1cc(Br)ccc1n2-c1ccccc1 ; OB(O)c1ccc2c(c1)c1ccccc1n2-c1ccccc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

Compound 1-2 was synthesized in the same manner as in Synthesis Example 1, except that in Synthesis Example 2, Compound 1-2-A and Compound 1-2-B (102.6 mmol) were used instead of Compound 1-1-A and Compound 1-1-B (51.3 mmol), respectively.

### Synthesis Example 3

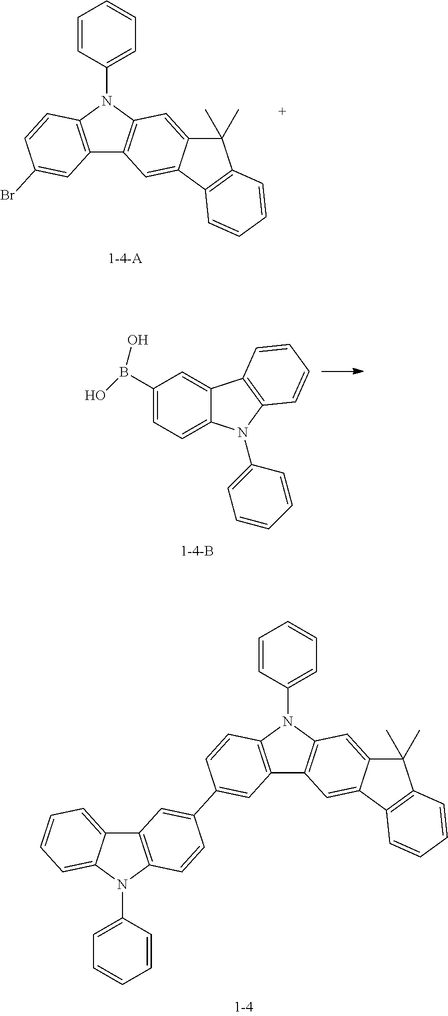 [SMILES: CC1(C)c2ccccc2-c2cc3c4cc(Br)ccc4n(-c4ccccc4)c3cc21 ; OB(O)c1ccc2c(c1)c1ccccc1n2-c1ccccc1 ; CC1(C)c2ccccc2-c2cc3c4cc(-c5ccc6c(c5)c5ccccc5n6-c5ccccc5)ccc4n(-c4ccccc4)c3cc21]

Compound 1-4 was synthesized in the same manner as in Synthesis Example 1, except that in Synthesis Example 3, Compound 1-4-A and Compound 1-4-B were used instead of Compound 1-1-A and Compound 1-1-B, respectively.

### Synthesis Example 4

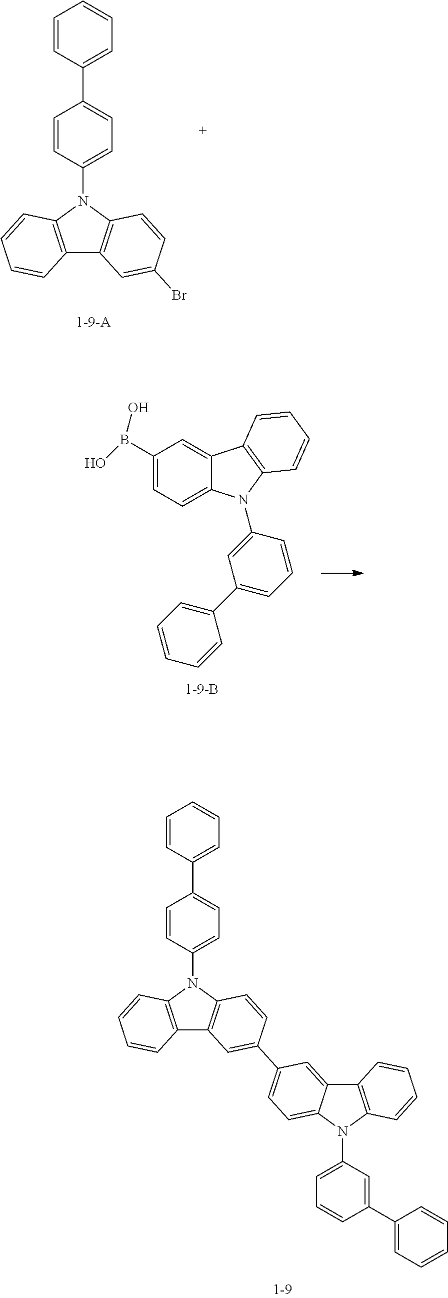 [SMILES: Brc1ccc2c(c1)c1ccccc1n2-c1ccc(-c2ccccc2)cc1 ; OB(O)c1ccc2c(c1)c1ccccc1n2-c1cccc(-c2ccccc2)c1 ; c1ccc(-c2ccc(-n3c4ccccc4c4cc(-c5ccc6c(c5)c5ccccc5n6-c5cccc(-c6ccccc6)c5)ccc43)cc2)cc1]

Compound 1-9 was synthesized in the same manner as in Synthesis Example 1, except that in Synthesis Example 4, Compound 1-9-A and Compound 1-9-B were used instead of Compound 1-1-A and Compound 1-1-B, respectively.

### Synthesis Example 5

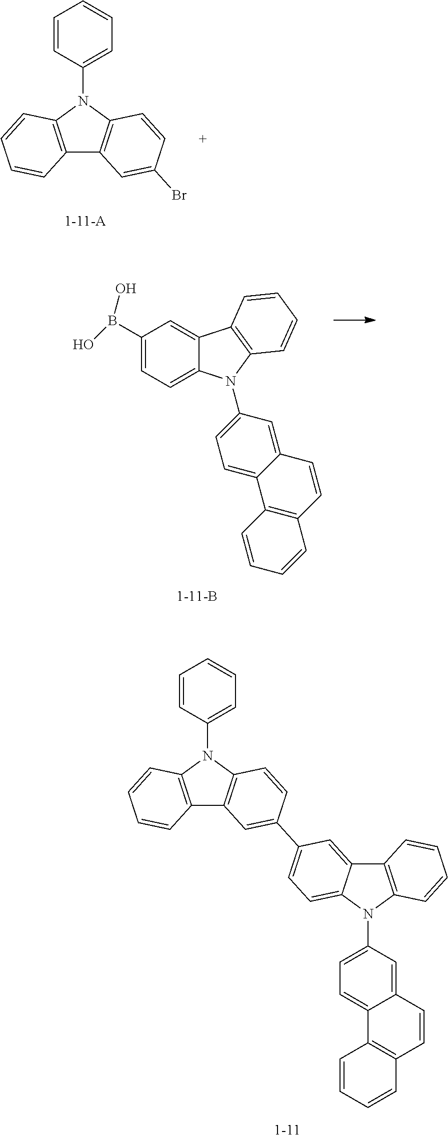 [SMILES: Brc1ccc2c(c1)c1ccccc1n2-c1ccccc1 ; OB(O)c1ccc2c(c1)c1ccccc1n2-c1ccc2c(ccc3ccccc32)c1 ; c1ccc(-n2c3ccccc3c3cc(-c4ccc5c(c4)c4ccccc4n5-c4ccc5c(ccc6ccccc65)c4)ccc32)cc1]

Compound 1-11 was synthesized in the same manner as in Synthesis Example 1, except that in Synthesis Example 5, Compound 1-11-A and Compound 1-11-B were used instead of Compound 1-1-A and Compound 1-1-B, respectively.

### Synthesis Example 6

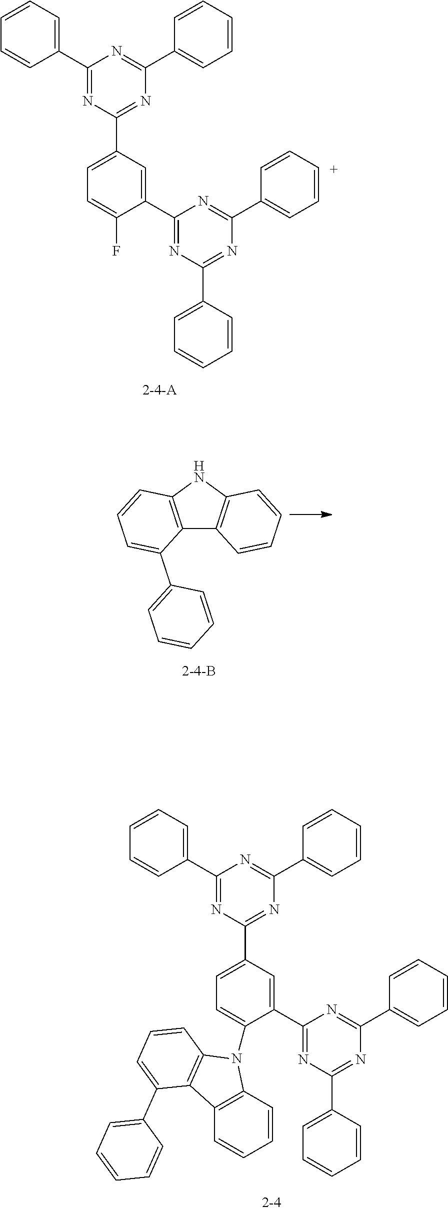 [SMILES: Fc1ccc(-c2nc(-c3ccccc3)nc(-c3ccccc3)n2)cc1-c1nc(C2=CC=[C+]C=C2)nc(-c2ccccc2)n1 ; c1ccc(-c2cccc3[nH]c4ccccc4c23)cc1 ; c1ccc(-c2nc(-c3ccccc3)nc(-c3ccc(-n4c5ccccc5c5c(-c6ccccc6)cccc54)c(-c4nc(-c5ccccc5)nc(-c5ccccc5)n4)c3)n2)cc1]

K₂CO₃ (4.91 mmol) and 2-4-B (3.93 mmol) were put into N-methyl pyrrolidone (NMP), and the resulting mixture was stirred at room temperature for 1 hour. Compound 2-4-A (0.655 mmol) was added thereto. This mixture was stirred at 180° C. for 18 hours. The mixture was again stirred at 195° C. for 11 hours. The reaction mixture was quenched with H₂O, the precipitated product was filtered, and then washed with methanol (MeOH). The filtered product was purified again by silica gel column chromatography (hexane:toluene=1:1) to obtain Compound 2-4 (0.422 mmol) as a white powder at a yield of 64%.

### Synthesis Example 7

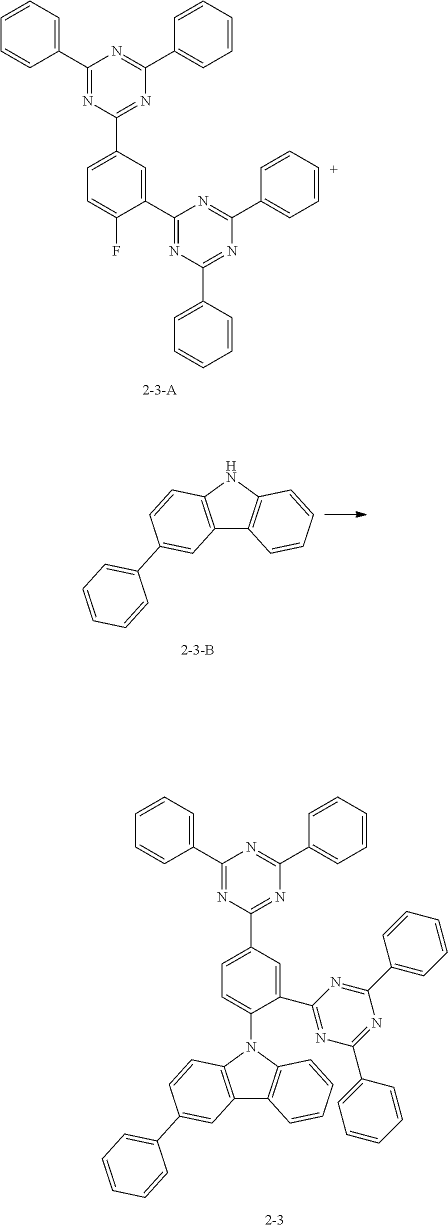 [SMILES: Fc1ccc(-c2nc(-c3ccccc3)nc(-c3ccccc3)n2)cc1-c1nc(C2=CC=[C+]C=C2)nc(-c2ccccc2)n1 ; c1ccc(-c2ccc3[nH]c4ccccc4c3c2)cc1 ; c1ccc(-c2ccc3c(c2)c2ccccc2n3-c2ccc(-c3nc(-c4ccccc4)nc(-c4ccccc4)n3)cc2-c2nc(-c3ccccc3)nc(-c3ccccc3)n2)cc1]

Compound 2-3 was synthesized in the same manner as in Synthesis Example 6, except that in Synthesis Example 7, Compound 2-3-A and Compound 2-3-B were used instead of Compound 2-4-A and Compound 2-4-B, respectively.

### Synthesis Example 8

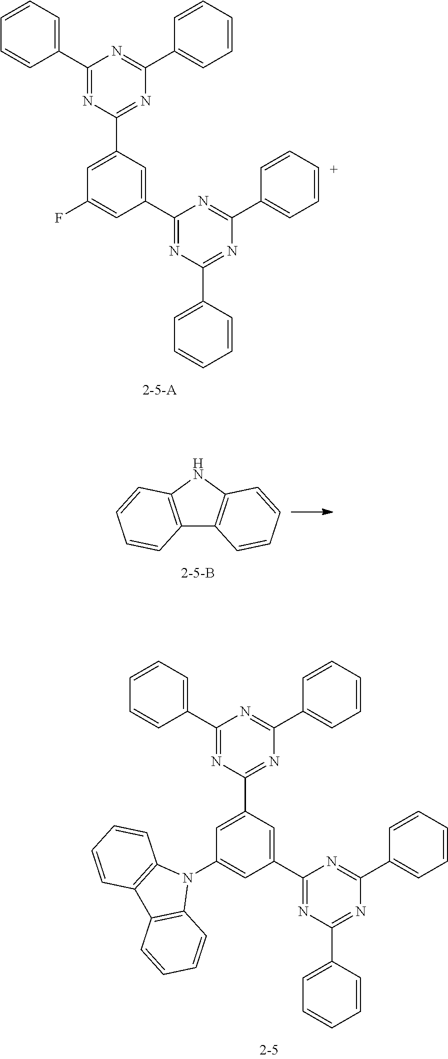 [SMILES: Fc1cc(-c2nc(C3=CC=[C+]C=C3)nc(-c3ccccc3)n2)cc(-c2nc(-c3ccccc3)nc(-c3ccccc3)n2)c1 ; c1ccc2c(c1)[nH]c1ccccc12 ; c1ccc(-c2nc(-c3ccccc3)nc(-c3cc(-c4nc(-c5ccccc5)nc(-c5ccccc5)n4)cc(-n4c5ccccc5c5ccccc54)c3)n2)cc1]

Compound 2-5 was synthesized in the same manner as in Synthesis Example 6, except that in Synthesis Example 8, Compound 2-5-A and Compound 2-5-B were used instead of Compound 2-4-A and Compound 2-4-B, respectively.

### Synthesis Example 9

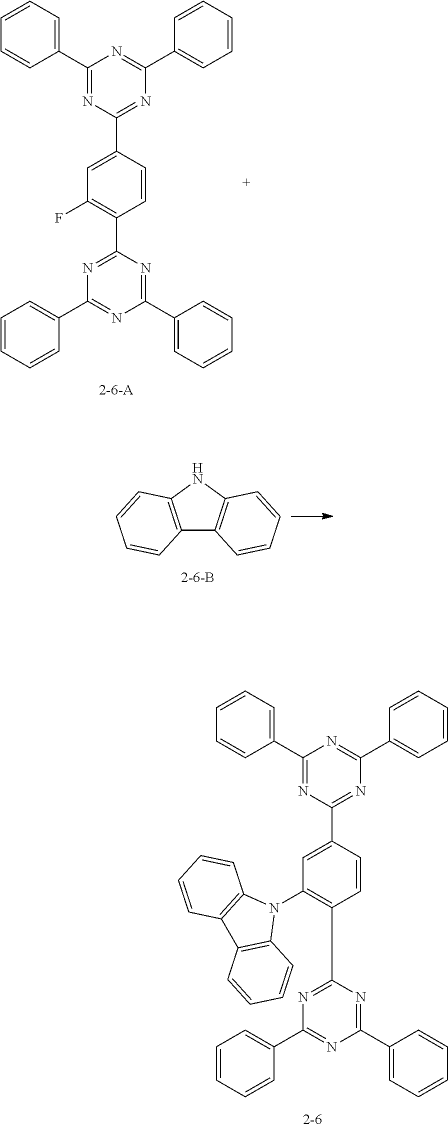 [SMILES: Fc1cc(-c2nc(-c3ccccc3)nc(-c3ccccc3)n2)ccc1-c1nc(-c2ccccc2)nc(-c2ccccc2)n1 ; c1ccc2c(c1)[nH]c1ccccc12 ; c1ccc(-c2nc(-c3ccccc3)nc(-c3ccc(-c4nc(-c5ccccc5)nc(-c5ccccc5)n4)c(-n4c5ccccc5c5ccccc54)c3)n2)cc1]

Compound 2-6 was synthesized in the same manner as in Synthesis Example 6, except that in Synthesis Example 9, Compound 2-6-A and Compound 2-6-B were used instead of Compound 2-4-A and Compound 2-4-B, respectively.

### Synthesis Example 10

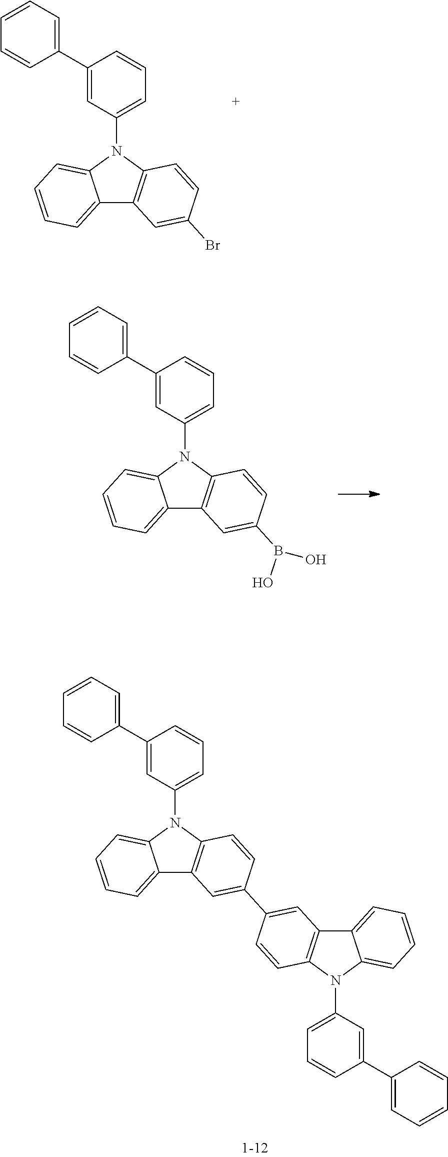 [SMILES: Brc1ccc2c(c1)c1ccccc1n2-c1cccc(-c2ccccc2)c1 ; OB(O)c1ccc2c(c1)c1ccccc1n2-c1cccc(-c2ccccc2)c1 ; c1ccc(-c2cccc(-n3c4ccccc4c4cc(-c5ccc6c(c5)c5ccccc5n6-c5cccc(-c6ccccc6)c5)ccc43)c2)cc1]

After 9-([1,1′-biphenyl]-3-yl)-3-bromo-9H-carbazole) (20.0 g, 50.4 mmol) and (9-([1,1′-biphenyl]-3-yl)-9H-carbazol-3-yl)boronic acid (18.3 g, 50.4 mmol) were dispersed in tetrahydrofuran (250 ml), a 2 M aqueous potassium carbonate solution (aq. K₂CO₃) (50.4 ml, 100.7 mmol) was added thereto, tetrakistriphenyl-phosphinepalladium [Pd(PPh₃)₄] (1.7 g, 3 mol %) was put thereinto, and then the resulting mixture was stirred and refluxed for 4 hours. The temperature was lowered to room temperature and the produced solid was filtered. The filtered solid was recrystallized with chloroform and ethyl acetate, filtered, and then dried to prepare Compound 1-12 (21.5 g, yield 67%; MS:[M+H]⁺=637).

### Comparative Example 1

As an anode, a substrate on which Ag/ITO were deposited to have a thickness of 100 nm/5 nm was cut into a size of 100 mm×100 mm×0.5 mm, put into distilled water in which a detergent was dissolved, and washed with ultrasonic waves. A product manufactured by Fischer Co. was used as the detergent, and distilled water twice filtered using a filter manufactured by Millipore Co. was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

The following compound HTL1 was thermally vacuum-deposited to have a thickness of 100 Å on the anode thus prepared, and co-deposited with compound PD1 (2 wt %), thereby forming a hole injection layer (HIL1). (Deposition rate: 1 Å/sec(HTL1), 0.3 Å/sec (PD1)):

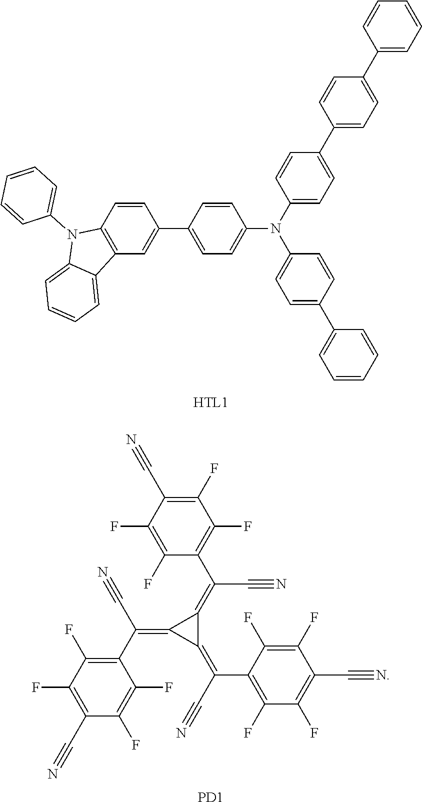 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD1"]

Only HTL1 was vacuum-deposited to have a thickness of 1100 Å thereon, thereby forming a first hole transport layer (HTL1), and the following compound HT-A was vacuum-deposited to have a thickness of 300 Å, thereby forming a second hole transport layer (HTL2) (Deposition rate: 2 Å/sec):

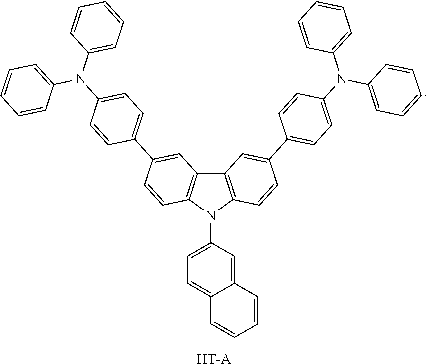

And then, the following compound EBL1 was vacuum-deposited to have a thickness of 150 Å, thereby forming an electron blocking layer (EBL) (Deposition rate: 1 Å/sec):

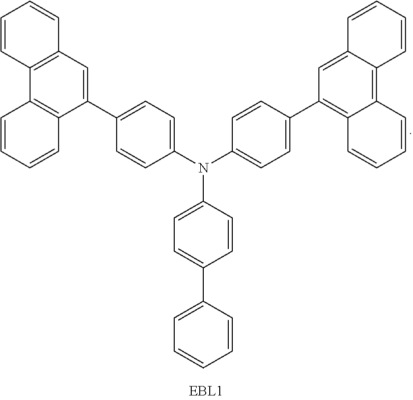 [SMILES: c1ccc(-c2ccc(N(c3ccc(-c4cc5ccccc5c5ccccc45)cc3)c3ccc(-c4cc5ccccc5c5ccccc45)cc3)cc2)cc1]

The following compound P-Host A, which is a P-type host, and Compound N-Host A, which is an N-type host (a weight ratio of P-Host A:N-Host A=6:4) as a host, and the following iridium compound (GD) (6 wt %) as a dopant were vacuum-deposited to have a thickness of 360 Å thereon, thereby forming a light emitting layer (EML) (Deposition rate: 1.2 Å/sec (P-Host), 0.8 Å/sec (N-Host A) 0.6 Å/sec (GD)):

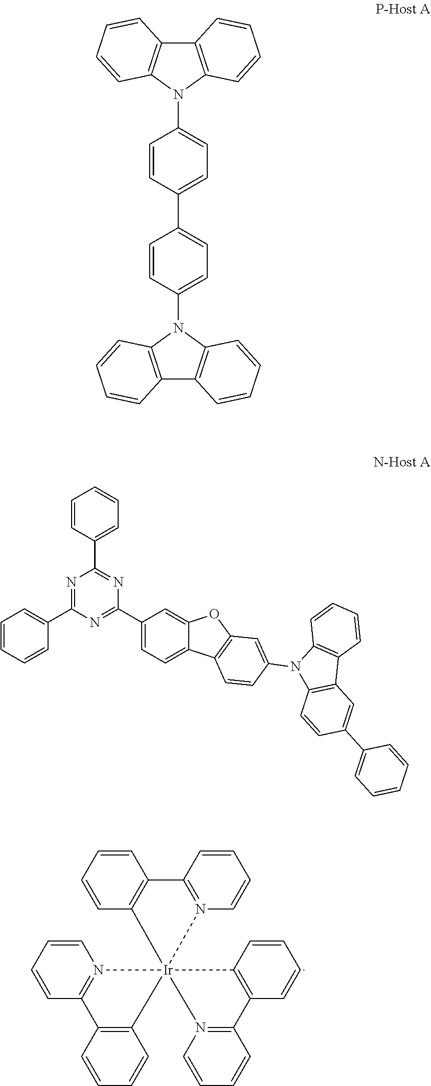

Thereafter, the following compound HBL1 was vacuum-deposited to have a thickness of 30 Å, thereby forming a hole blocking layer (HBL), and compounds ETL1 and Liq were vacuum-deposited at a weight ratio of 2:1, thereby forming an electron transport layer (ETL) (Deposition rate: 1 Å/sec (HBL1), 1.2 Å/sec (ETL1), 0.6 Å/sec (Liq)):

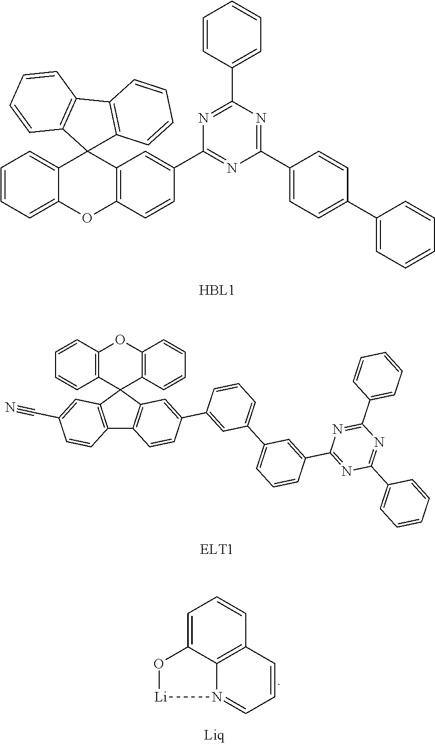

Sequentially, after ytterbium (Yb) and lithium fluoride (LiF) were deposited to have a thickness of 20 A to form a film as an electron injection layer (EIL), magnesium and silver (1:4) were used to form a cathode having a thickness of 120 Å, and then the following compound CPL was deposited to have a thickness of 600 Åas a capping layer, thereby completing a device (Deposition rate: 0.3 Å/sec (Yb, LiF), 1 Å/sec (Ag), 0.1 Å/sec (Mg)):

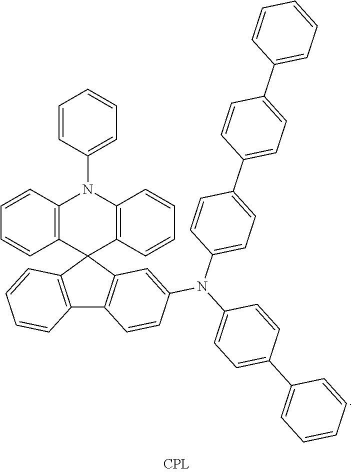 [SMILES: c1ccc(-c2ccc(-c3ccc(N(c4ccc(-c5ccccc5)cc4)c4ccc5c(c4)C4(c6ccccc6-5)c5ccccc5N(c5ccccc5)c5ccccc54)cc3)cc2)cc1]

### Examples 1 to 9 and Comparative Examples 2 to 7

Devices of Examples 1 to 9 and Comparative Examples 2 to 7 were each manufactured in the same manner as in Comparative Example 1, except that in Comparative Example 1, the compounds shown in Table 1 were each used as a P-type host and an N-type host.

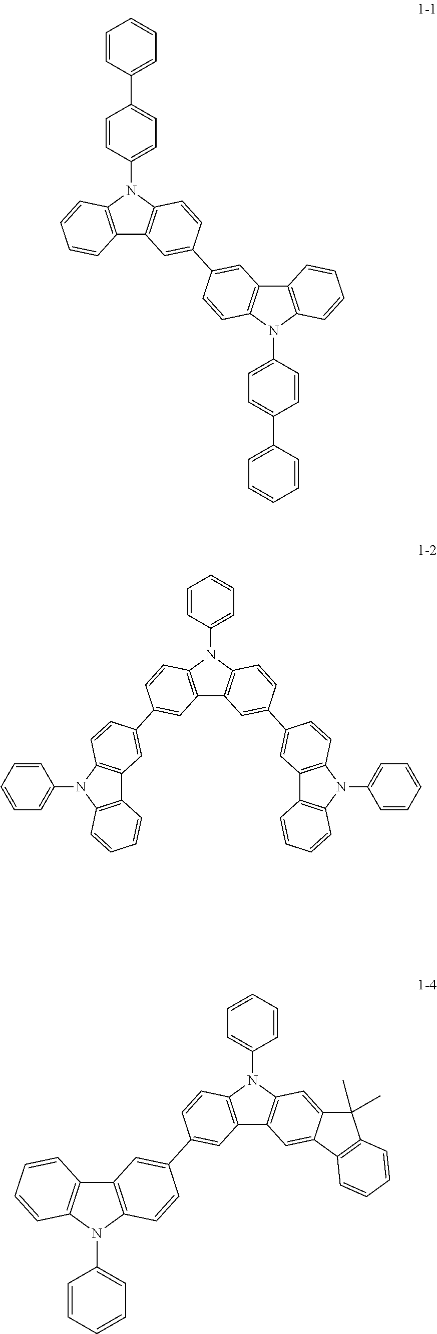 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; CC1(C)c2ccccc2-c2cc3c4cc(-c5ccc6c(c5)c5ccccc5n6-c5ccccc5)ccc4n(-c4ccccc4)c3cc21]

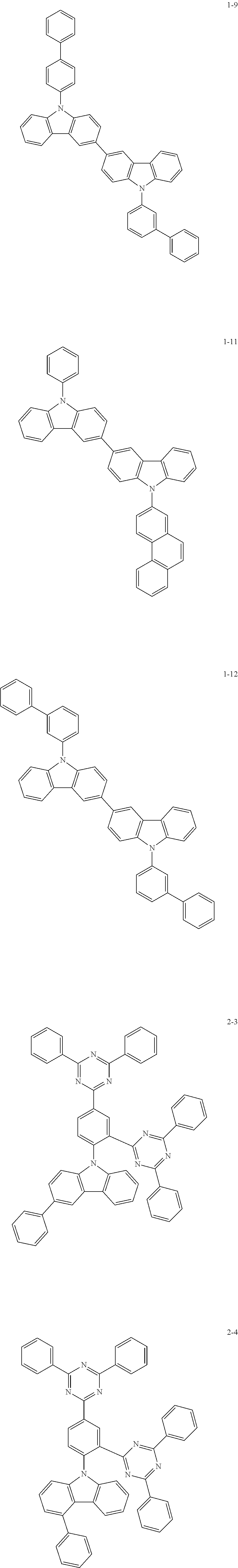 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4ccccc4n5-c4ccc5c(ccc6ccccc65)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc3c(c2)c2ccccc2n3-c2ccc(-c3nc(-c4ccccc4)nc(-c4ccccc4)n3)cc2-c2nc(-c3ccccc3)nc(-c3ccccc3)n2)cc1 ; c1ccc(-c2nc(-c3ccccc3)nc(-c3ccc(-n4c5ccccc5c5c(-c6ccccc6)cccc54)c(-c4nc(-c5ccccc5)nc(-c5ccccc5)n4)c3)n2)cc1]

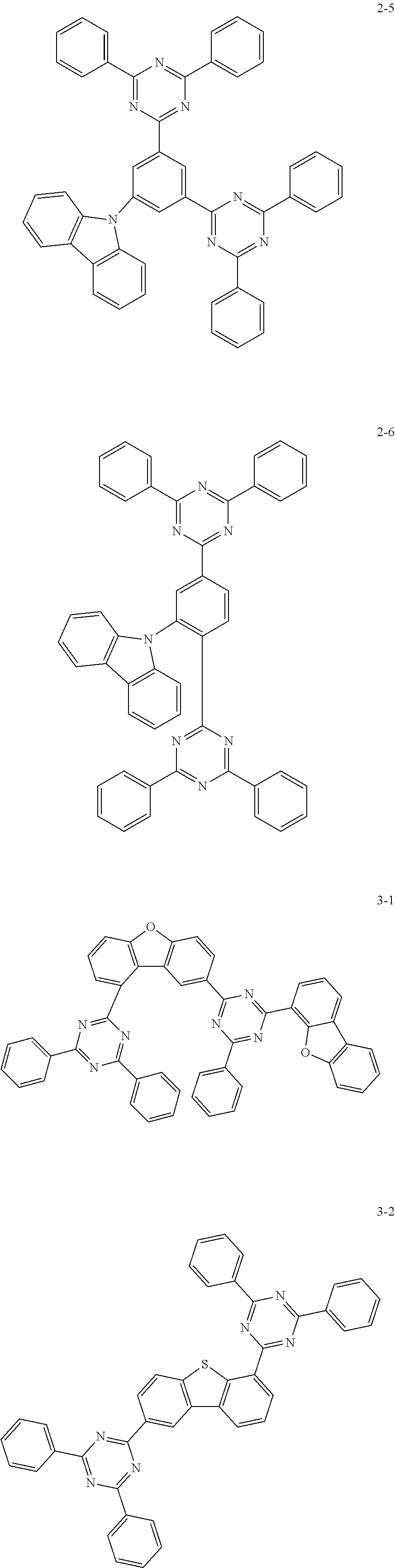 [SMILES: c1ccc(-c2nc(-c3ccccc3)nc(-c3cc(-c4nc(-c5ccccc5)nc(-c5ccccc5)n4)cc(-n4c5ccccc5c5ccccc54)c3)n2)cc1 ; c1ccc(-c2nc(-c3ccccc3)nc(-c3ccc(-c4nc(-c5ccccc5)nc(-c5ccccc5)n4)c(-n4c5ccccc5c5ccccc54)c3)n2)cc1 ; c1ccc(-c2nc(-c3ccc4oc5cccc(-c6nc(-c7ccccc7)nc(-c7ccccc7)n6)c5c4c3)nc(-c3cccc4c3oc3ccccc34)n2)cc1 ; c1ccc(-c2nc(-c3ccccc3)nc(-c3ccc4sc5c(-c6nc(-c7ccccc7)nc(-c7ccccc7)n6)cccc5c4c3)n2)cc1]

### Experimental Examples

Experiments were conducted by evaporating OLED organic compounds in a vacuum chamber of 1.0E⁻⁷ or higher. After the encapsulation process was completed, baking was performed in an oven at 110° C. for 40 minutes. In this case, the aging effect of the organic thin film could be seen by baking, so that a stable upper light emitting organic EL device could be obtained.

The IVL characteristics of the completely baked organic EL device were measured by utilizing a PR-670 IVL measuring instrument manufactured by Photo Research Inc., and the life time (LT) of the upper organic EL device was measured by the photo diode method based on 20,000 nit through a M6000 product manufactured by McScience, so that the time required for the luminance to decrease from the initial luminance to 95% was shown as T95.

TABLE 1

| | | | | | LT@20000 |
|---|---|---|---|---|---|
| | | | | | nit/cm² |
| | P-type host | N-type host | V | cd/A | (T95) |
| Comparative | P-Host A | N-Host A | 4.30 | 122.3 | 163.5 |
| Example 1 | | | | | |
| Comparative | 1-1 | N-Host A | 4.34 | 138.5 | 151.5 |
| Example 2 | | | | | |
| Comparative | P-Host A | 2-3 | 4.21 | 129.6 | 187.2 |
| Example 3 | | | | | |
| Example 1 | 1-9 | 2-3 | 4.66 | 135.9 | 555.5 |
| Example 2 | 1-9 | 2-4 | 4.09 | 137.1 | 534.2 |
| Example 3 | 1-1 | 2-5 | 4.18 | 136.4 | 371.4 |
| Example 4 | 1-2 | 2-6 | 4.29 | 138.3 | 379.8 |
| Comparative | 1-4 | 3-1 | 4.42 | 128.1 | 353.2 |
| Example 4 | | | | | |
| Comparative | 1-11 | 3-2 | 4.55 | 129.6 | 468.0 |
| Example 5 | | | | | |
| Example 5 | 1-11 | 2-3 | 4.19 | 130 | 190.2 |
| Example 6 | 1-12 | 2-3 | 4.45 | 137.2 | 556.3 |
| Example 7 | 1-12 | 2-4 | 3.87 | 140.2 | 564.8 |
| Example 8 | 1-12 | 2-5 | 3.87 | 156.9 | 392.1 |
| Example 9 | 1-12 | 2-6 | 4.06 | 128.9 | 400.3 |
| Comparative | 1-12 | 3-1 | 4.22 | 158.7 | 348.1 |
| Example 6 | | | | | |
| Comparative | 1-12 | 3-2 | 4.32 | 136.8 | 356.5 |
| Example 7 | | | | | |

* V: Driving voltage
* cd/A: Efficiency

From the results in Table 1, it can be confirmed that Examples 1 to 4 satisfy low voltage, high efficiency, or long service life compared to Comparative Examples 1 to 5.

It can be confirmed that in the case of Example 1, the service life of 100 hours of Comparative Examples 1 to 3 is dramatically extended to 500 hours, even though the driving voltage is increased by 0.3 V due to the combination of hosts described in the present specification.

It can be confirmed that the efficiencies of Examples 1 to 4 are maintained at levels similar to those of the Comparative Examples, but the driving voltage is lowered and the service life is greatly improved.

As shown in Comparative Example 5, it can be confirmed that the combination using the N-type host similar to Chemical Formula 2 of the present specification has a longer life, but a higher voltage and a lower efficiency.

It can be confirmed that when comparing Example 5 in which Compound 2-3 is used and Comparative Example 5 in which Compound 3-2 is used, as the N-type host, Compound 3-2 increases the service life by attaching triazine, which has a large amount of electrons, to a material having a certain amount of holes, such as dibenzofuran or dibenzothiophene to control the flow of electrons, but the electrons cannot flow smoothly, and as a result, the voltage is increased.

Compounds 3-1 and 3-2 have a large volume due to a higher molecular weight than that of Compound 2-3, and the sublimation of the material causes problems in mass production during the mass production of a panel. Specifically, the preferred material should be melted or remain powdery when heated, but Compounds 3-1 and 3-2 will harden to the top of the crucible like hard cotton, so that the material will not sublimate.

### Example 10, Example 11, Comparative Example 8, and Comparative Example 9

Devices of Example 10, Example 11, Comparative Example 8, and Comparative Example 9 were each manufactured in the same manner as in Example 7, except that in Example 7 the compounds shown in Table 2 were each used as a P-type host, an N-type host, an electron blocking layer, and a capping layer.

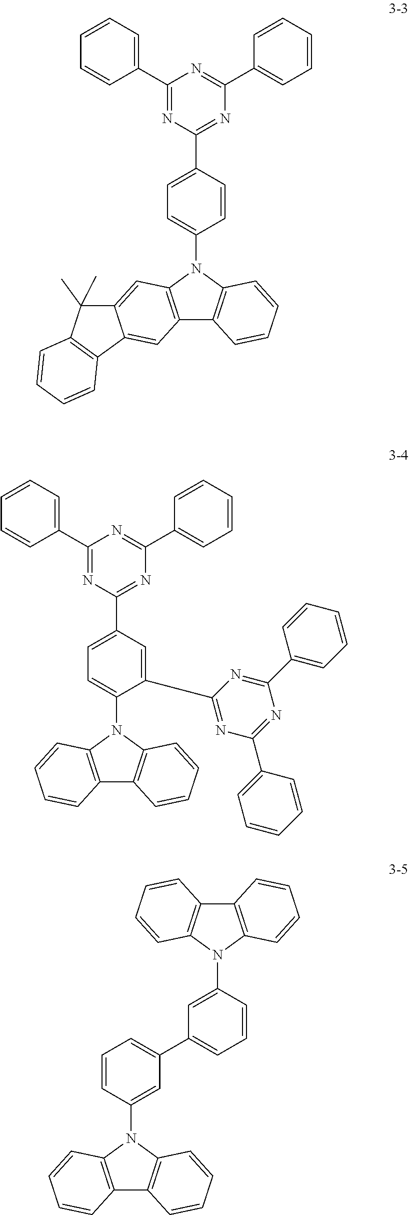 [SMILES: CC1(C)c2ccccc2-c2cc3c4ccccc4n(-c4ccc(-c5nc(-c6ccccc6)nc(-c6ccccc6)n5)cc4)c3cc21 ; c1ccc(-c2nc(-c3ccccc3)nc(-c3ccc(-n4c5ccccc5c5ccccc54)c(-c4nc(-c5ccccc5)nc(-c5ccccc5)n4)c3)n2)cc1 ; c1cc(-c2cccc(-n3c4ccccc4c4ccccc43)c2)cc(-n2c3ccccc3c3ccccc32)c1]

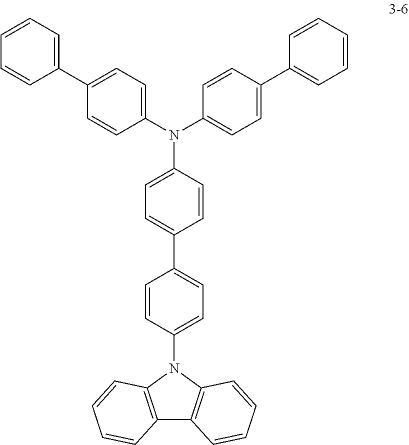 [SMILES: c1ccc(-c2ccc(N(c3ccc(-c4ccccc4)cc3)c3ccc(-c4ccc(-n5c6ccccc6c6ccccc65)cc4)cc3)cc2)cc1]

Experiments were conducted by evaporating OLED organic compounds in a vacuum chamber of 1.0E⁻⁷ or higher. After the encapsulation process was completed, baking was performed in an oven at 110° C. for 40 minutes. In this case, the aging effect of the organic thin film could be seen by baking, so that a stable upper light emitting organic EL device could be obtained.

The IVL characteristics of the completely baked organic EL device were measured by utilizing a PR-670 IVL measuring instrument manufactured by Photo Research Inc., and the life time (LT) of the upper organic EL device was measured by the photo diode method based on 20,000 nit through a M6000 product manufactured by McScience, so that the time required for the luminance to decrease from the initial luminance to 95% was shown as T95.

TABLE 2

| | P- | N- | | | | | LT@20000 |
|---|---|---|---|---|---|---|---|
| | type | type | | Capping | | | nit/cm² |
| | host | host | EBL | layer | V | cd/A | (195) |
| Example 7 | 1-12 | 2-4 | EBL1 | CPL | 3.87 | 140.2 | 564.8 |
| Example 10 | 1-12 | 2-4 | 3-5 | CPL | 3.95 | 136.4 | 478.2 |
| Example 11 | 1-12 | 2-4 | 3-6 | CPL | 4.58 | 116.7 | 382.2 |
| Comparative | 1-12 | 3-3 | 3-6 | — | 4.1 | 120.6 | 465.7 |
| Example 8 | | | | | | | |
| Comparative | 3-5 | 3-4 | 3-5 | — | 4.82 | 105.8 | 220.4 |
| Example 9 | | | | | | | |

It was confirmed that Examples 10 and 11 using Compounds 3-6 and 3-5, respectively, had a shorter service life than Example 7 using compound EBL1, as the hole blocking layer. It is determined that because the hole transport layer deteriorates using Compound 3-5 or Compound 3-6, which has lower hole transport characteristics than compound EBL1, the service life is shortened to some extent.

As the N-type host of the present application is composed of two triazines, the amount of electrons becomes relatively too dominant over holes, so that the energy balance of electrons and holes does not match in the light emitting layer, but the balance lost in the light emitting layer is restored using compound EBL1 rich in holes in the EBL layer adjacent to the light emitting layer. As a result, Example 7 in which the balance was created using compound EBL1 showed better performance than Comparative Example 8 in which Compound 3-3 having one triazine was used.

1. An organic light emitting device comprising:
a first electrode;
a second electrode; and
an organic material layer having one or more layers provided between the first electrode and the second electrode,
wherein the organic material layer comprises a light emitting layer comprising a compound of the following Chemical Formula 1 and a compound of the following Chemical Formula 2: 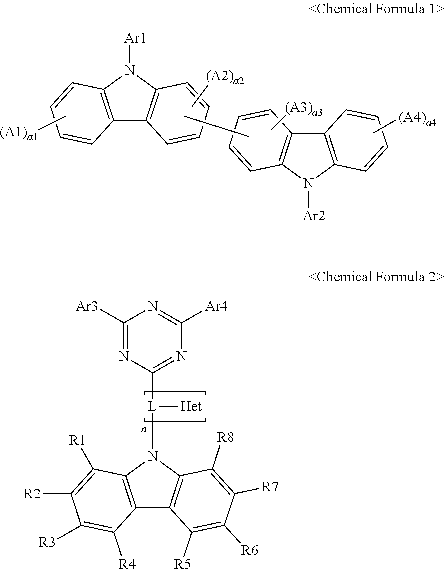
wherein in Chemical Formulae 1 and 2:
Ar1 to Ar4 are the same as or different from each other, and are each independently a substituted or unsubstituted aryl group or a substituted or unsubstituted heterocyclic group;
A1 to A4 and R1 to R8 are the same as or different from each other, and are each independently hydrogen, deuterium, a substituted or unsubstituted aryl group, or a substituted or unsubstituted carbazole group, or are bonded to an adjacent group to form a substituted or unsubstituted
Het is a substituted or unsubstituted heterocyclic group having 5 to 20 carbon atoms, which comprises N, O, or S;
L is a trivalent aryl group having 5 to 20 carbon atoms;
a1 and a4 are each an integer from 1 to 4, and a2 and a3 are each an integer from 1 to 3;
when a1 to a4 are each an integer of 2 or higher, two or more substituents in the parenthesis are the same as or different from each other;
n is an integer of 0 or 1; and
when n is 0, N of the carbazole is directly bonded to the triazine.

2. The organic light emitting device of claim 1, wherein the compound of Chemical Formula 1 is the following Chemical Formula 1-1: 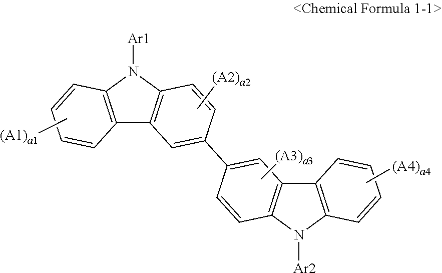
wherein in Chemical Formula 1-1, Ar1, Ar2, A1 to A4, and a1 to a4 are the same as definitions in Chemical Formula 1.

3. The organic light emitting device of claim 1, wherein the compound of Chemical Formula 1 is any one of the following Chemical Formulae 1-1-1 to 1-1-3: 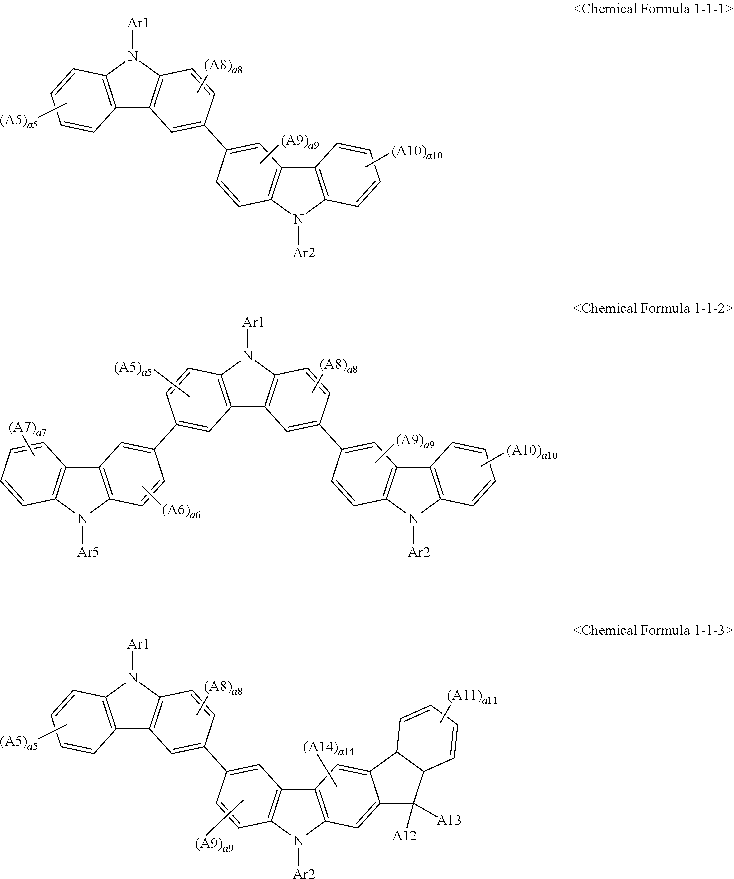
wherein in Chemical Formulae 1-1-1 to 1-1-3:
Ar1, Ar2, and Ar5 are the same as or different from each other, and are each independently a substituted or unsubstituted aryl group having 6 to 20 carbon atoms;
A5 to A11 and A14 are the same as or different from each other, and are each independently hydrogen, deuterium, or a substituted or unsubstituted aryl group;
A12 and A13 are the same as or different from each other, and are each independently a substituted or unsubstituted alkyl group or a substituted or unsubstituted aryl group;
a5, a6, a8, and a9 are each an integer from 1 to 3, and a7, a10, and a11 are each an integer from 1 to 4;
when a5 to a11 are each an integer of 2 or higher, two or more structures in the parenthesis are the same as or different from each other; and
a14 is 1 or 2, and when a14 is 2, structures in the parenthesis are the same as or different from each other.

4. The organic light emitting device of claim 1, where Ar1 and Ar2 are the same as or different from each other, and are each independently a substituted or unsubstituted phenyl group, a substituted or unsubstituted biphenyl group, a substituted or unsubstituted terphenyl group, a substituted or unsubstituted naphthyl group, or a substituted or unsubstituted phenanthrenyl group.

5. The organic light emitting device of claim 1, wherein the compound of Chemical Formula 2 is the following Chemical Formula 2-1: 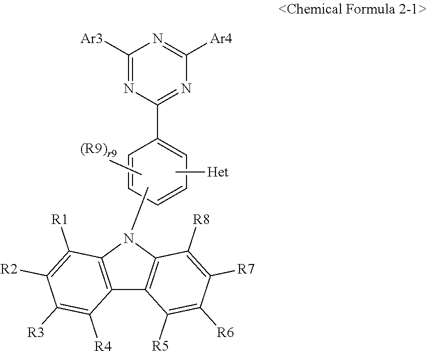
wherein in Chemical Formula 2-1:
Ar3, Ar4, and Het are the same as the definitions in Chemical Formula 2;
R1 to R9 are the same as or different from each other, and are each independently hydrogen, deuterium, or a substituted or unsubstituted aryl group; and
r9 is an integer from 1 to 3, and when r9 is an integer of 2 or higher, two or more structures in the parenthesis are the same as or different from each other.

6. The organic light emitting device of claim 1, wherein the compound of Chemical Formula 2 is the following Chemical Formula 2-2: 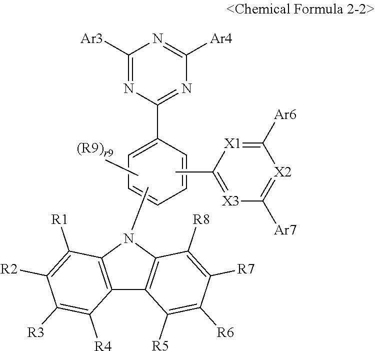
wherein in Chemical Formula 2-2:
Ar3, Ar4, Ar6, and Ar7 are the same as or different from each other, and are each independently a substituted or unsubstituted aryl group;
at least one of X1 to X3 is N, and the others are CH;
R1 to R9 are the same as or different from each other, and are each independently hydrogen, deuterium, or a substituted or unsubstituted aryl group; and
r9 is an integer from 1 to 3, and when r9 is an integer of 2 or higher, two or more structures in the parenthesis are the same as or different from each other.

7. The organic light emitting device of claim 6, wherein X1 to X3 are N.

8. The organic light emitting device of claim 1, wherein the compound of Chemical Formula 2 is any one of the following Chemical Formulae 2-2-1 to 2-2-3: 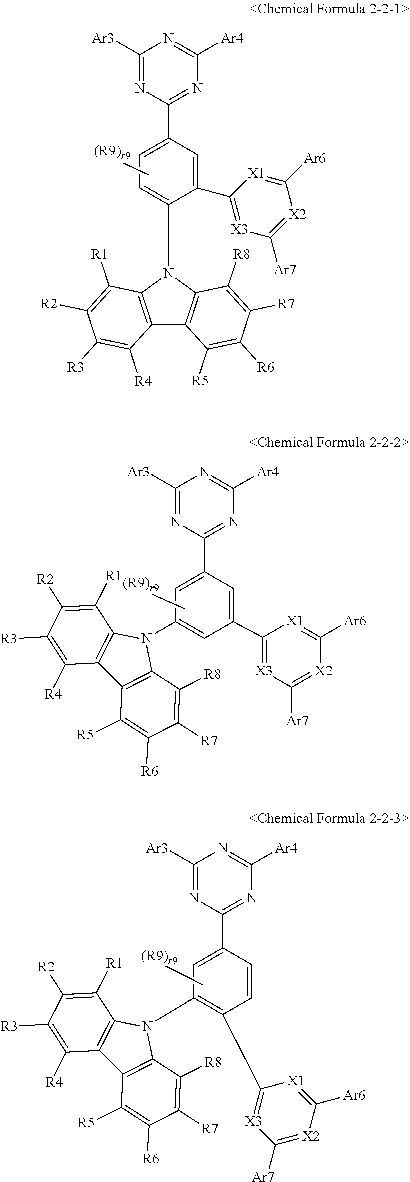
wherein in Chemical Formulae 2-2-1 to 2-2-3;
Ar3, Ar4, Ar6, and Ar7 are the same as or different from each other, and are each independently a substituted or unsubstituted aryl group;
at least one of X1 to X3 is N, and the others are CH;
R1 to R9 are the same as or different from each other, and are each independently hydrogen, deuterium, or a substituted or unsubstituted aryl group; and
r9 is an integer from 1 to 3, and when r9 is an integer of 2 or higher, two or more structures in the parenthesis are the same as or different from each other.

9. The organic light emitting device of claim 1, wherein the compound of Chemical Formula 1 is any one of the following compounds:

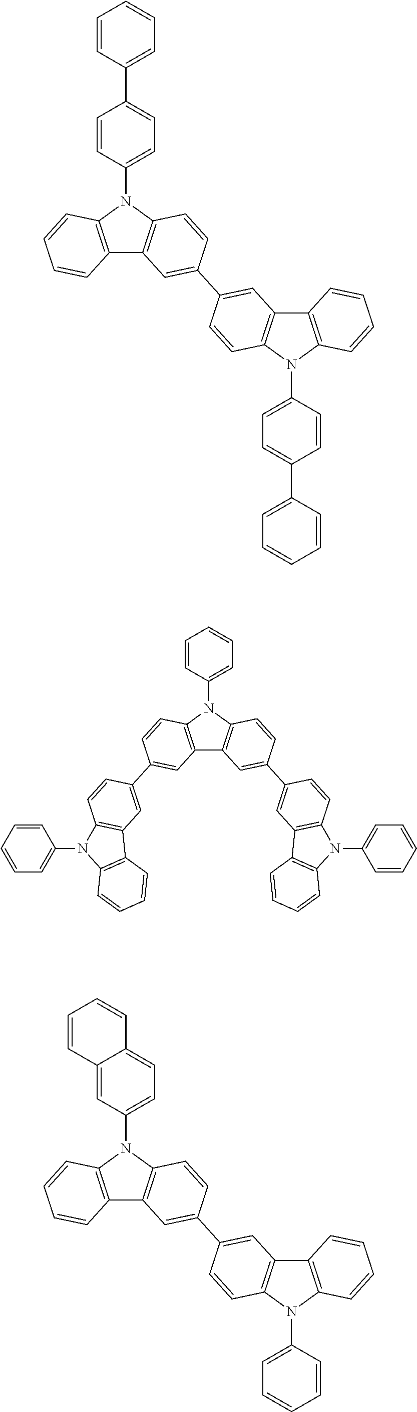 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccc5ccccc5c4)ccc32)cc1]

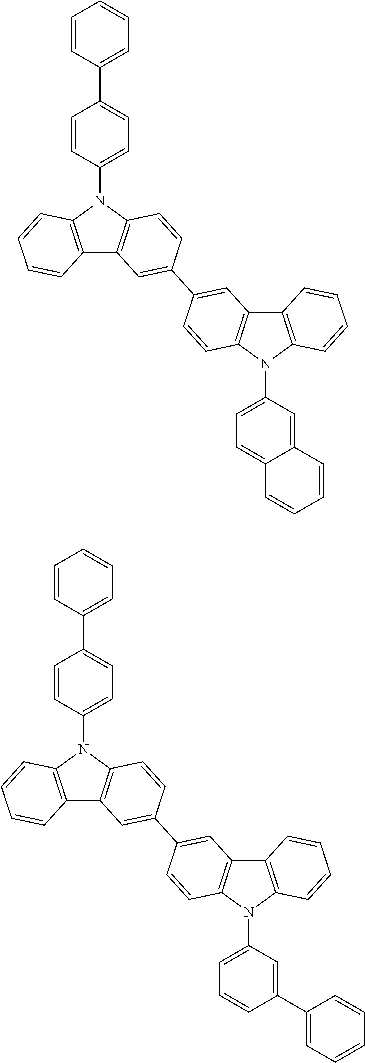 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

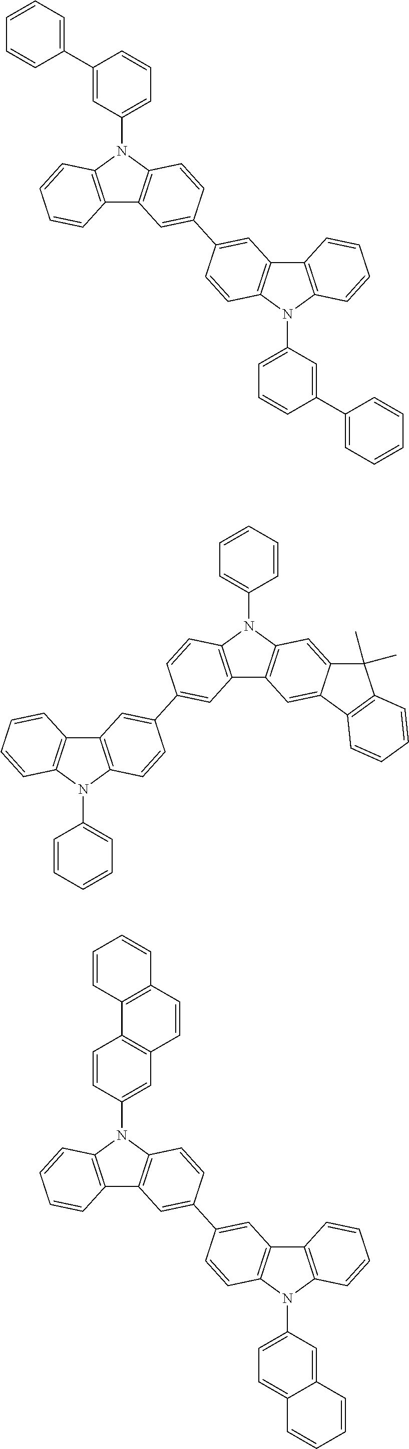 [SMILES: c1ccc(-c2cccc(-n3c4ccccc4c4cc(-c5ccc6c(c5)c5ccccc5n6-c5cccc(-c6ccccc6)c5)ccc43)c2)cc1 ; CC1(C)c2ccccc2-c2cc3c4cc(-c5ccc6c(c5)c5ccccc5n6-c5ccccc5)ccc4n(-c4ccccc4)c3cc21 ; c1ccc2cc(-n3c4ccccc4c4cc(-c5ccc6c(c5)c5ccccc5n6-c5ccc6c(ccc7ccccc76)c5)ccc43)ccc2c1]

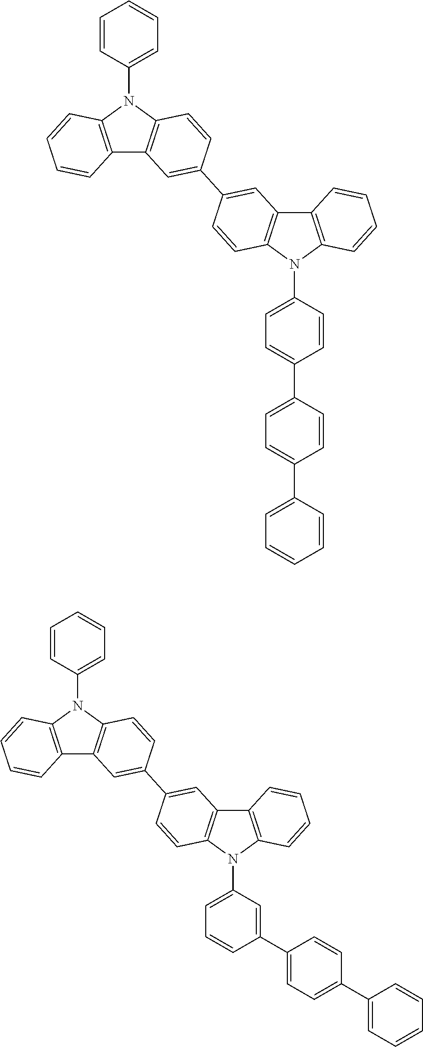 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

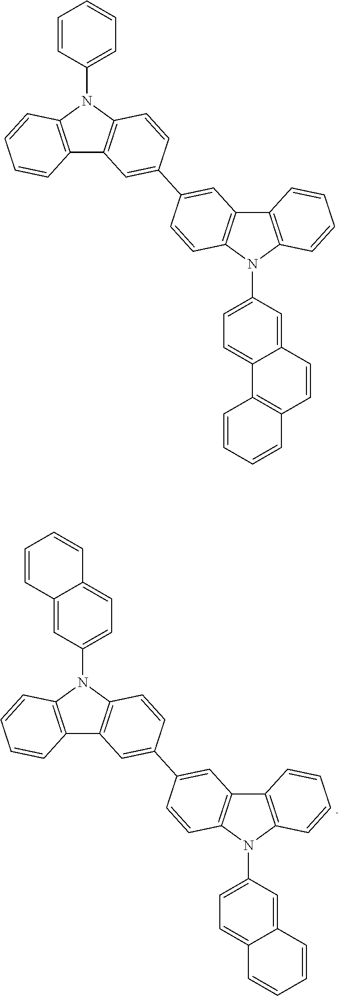 [SMILES: c1ccc(-n2c3ccccc3c3cc(-c4ccc5c(c4)c4ccccc4n5-c4ccc5c(ccc6ccccc65)c4)ccc32)cc1 ; c1ccc2cc(-n3c4ccccc4c4cc(-c5ccc6c(c5)c5ccccc5n6-c5ccc6ccccc6c5)ccc43)ccc2c1]

10. The organic light emitting device of claim 1, wherein the compound of Chemical Formula 2 is any one of the following compounds:

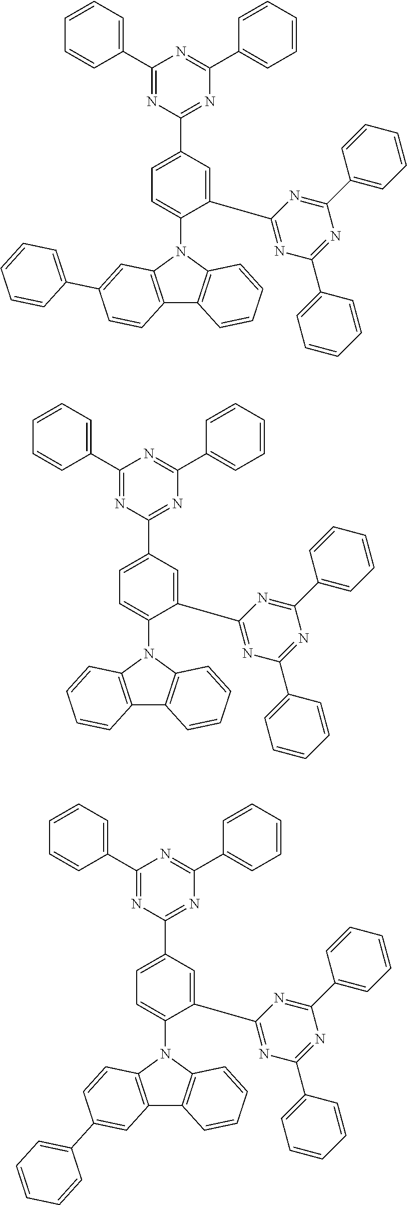 [SMILES: c1ccc(-c2ccc3c4ccccc4n(-c4ccc(-c5nc(-c6ccccc6)nc(-c6ccccc6)n5)cc4-c4nc(-c5ccccc5)nc(-c5ccccc5)n4)c3c2)cc1 ; c1ccc(-c2nc(-c3ccccc3)nc(-c3ccc(-n4c5ccccc5c5ccccc54)c(-c4nc(-c5ccccc5)nc(-c5ccccc5)n4)c3)n2)cc1 ; c1ccc(-c2ccc3c(c2)c2ccccc2n3-c2ccc(-c3nc(-c4ccccc4)nc(-c4ccccc4)n3)cc2-c2nc(-c3ccccc3)nc(-c3ccccc3)n2)cc1]

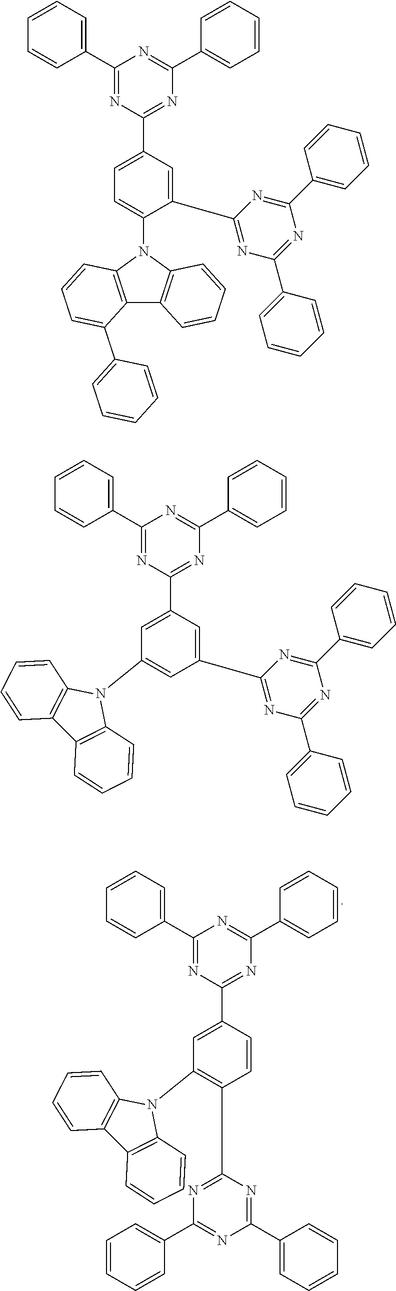 [SMILES: c1ccc(-c2nc(-c3ccccc3)nc(-c3ccc(-n4c5ccccc5c5c(-c6ccccc6)cccc54)c(-c4nc(-c5ccccc5)nc(-c5ccccc5)n4)c3)n2)cc1 ; c1ccc(-c2nc(-c3ccccc3)nc(-c3cc(-c4nc(-c5ccccc5)nc(-c5ccccc5)n4)cc(-n4c5ccccc5c5ccccc54)c3)n2)cc1 ; c1ccc(-c2nc(-c3ccccc3)nc(-c3ccc(-c4nc(-c5ccccc5)nc(-c5ccccc5)n4)c(-n4c5ccccc5c5ccccc54)c3)n2)cc1]

11. The organic light emitting device of claim 1, wherein the light emitting layer comprises a host and a dopant,
the host comprises the compound of Chemical Formula 1 and the compound of Chemical Formula 2, and
the dopant is a phosphorescent dopant.

12. The organic light emitting device of claim 11, wherein the phosphorescent dopant is any one of the following compounds: 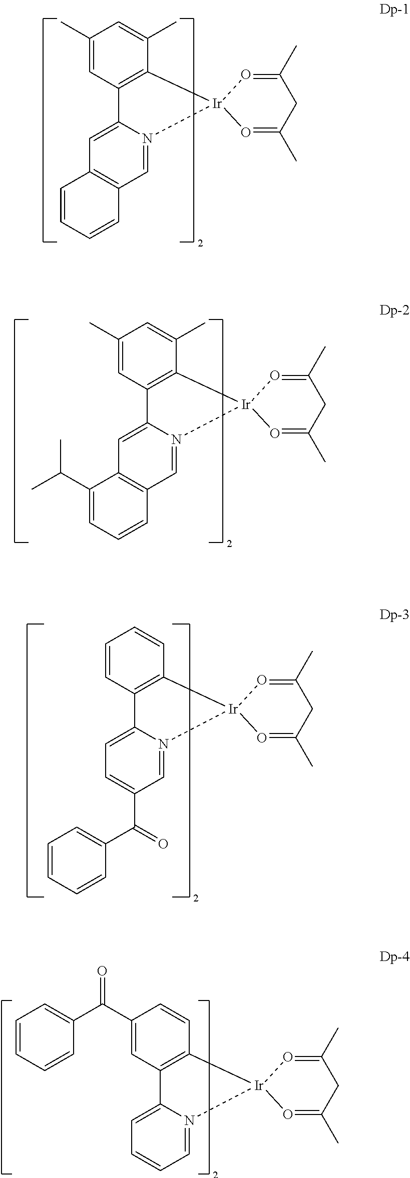 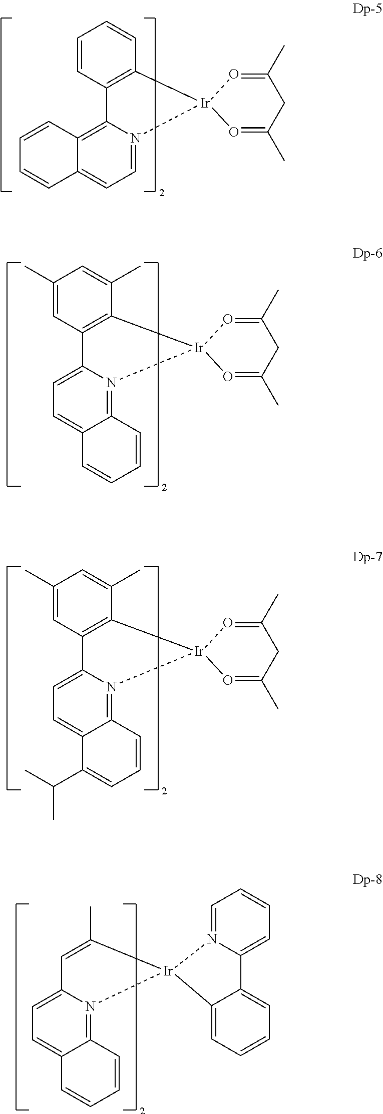 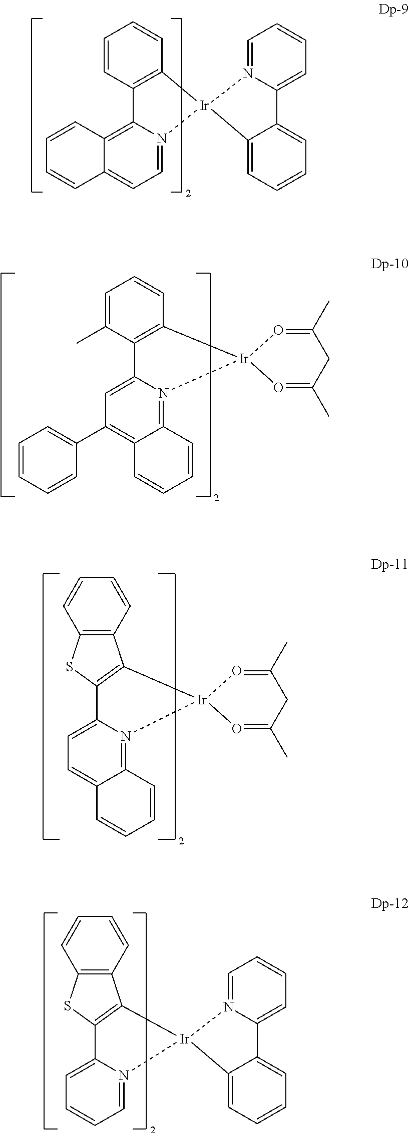 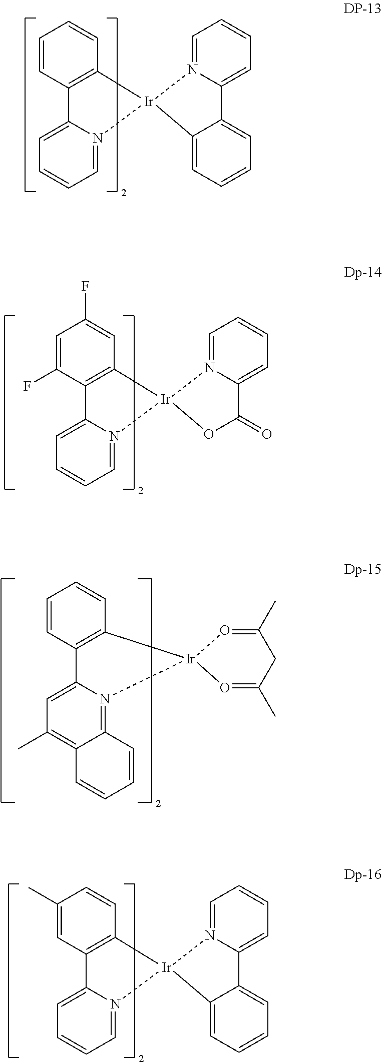 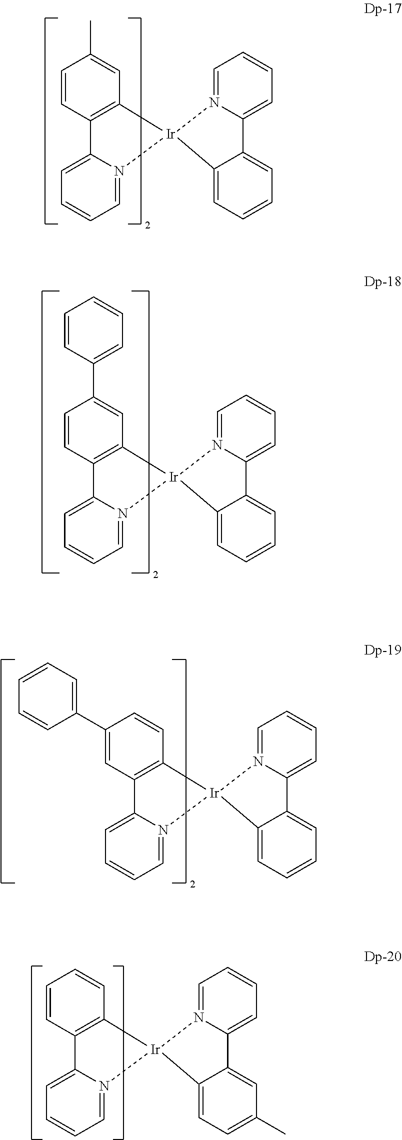 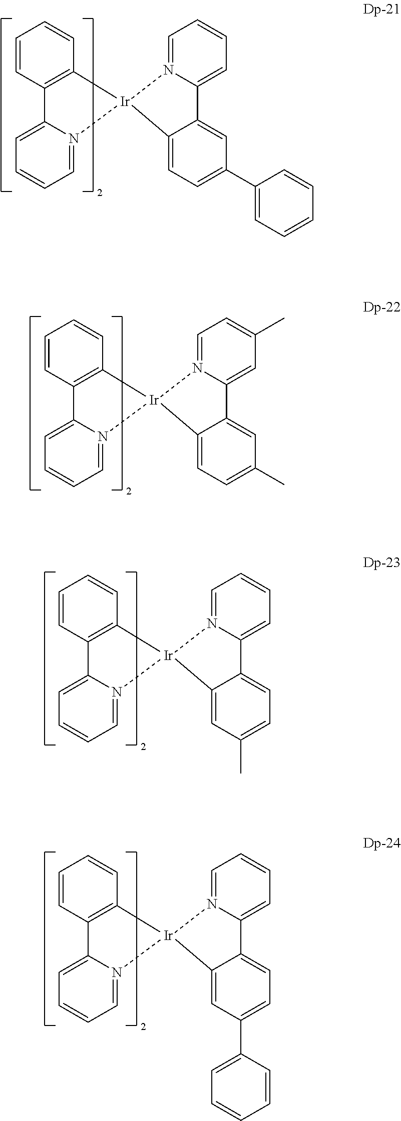 [chemical structure]
